SEQUENCE LISTING (20 sequences)

SEQ ID NO: 1 - DNA, 3023 bp, Homo sapiens
  CDS (89)..(2482)
caggtggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacggaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagataaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacacttaaaacctagaacttcaaaatgttcgtattctattcaaaaggaaaaatatatatatatatatatatatataaattaaaaaaaaaaaaaaaa

SEQ ID NO: 2 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDKSDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 3 - protein, 5 aa, Mus musculus
  VARIANT 2, 3: Xaa = Any Amino Acid
LXXLL

SEQ ID NO: 4 - DNA, 3066 bp, Mus musculus
  CDS (92)..(2482)
aattcggcacgaggttgcctgcatgagtgtgtgctgtgtgtcagagtggattggagttgaaaaagcttgactggcgtcattcgggagctggatggcttgggacatgtgcagccaagactctgtatggagtgacatagagtgtgctgctctggttggtgaggaccagcctctttgcccagatcttcctgaacttgacctttctgaacttgatgtgaatgacttggatacagacagctttctgggtggattgaagtggtgtagcgaccaatcggaaatcatatccaaccagtacaacaatgagcctgcgaacatatttgagaagatagatgaagagaatgaggcaaacttgctagcggtcctcacagagacactggacagtctccccgtggatgaagacggattgccctcatttgatgcactgacagatggagccgtgaccactgacaacgaggccagtccttcctccatgcctgacggcacccctccccctcaggaggcagaagagccgtctctacttaagaagctcttactggcaccagccaacactcagctcagctacaatgaatgcagcggtcttagcactcagaaccatgcagcaaaccacacccacaggatcagaacaaaccctgccattgttaagaccgagaattcatggagcaataaagcgaagagcatttgtcaacagcaaaagccacaaagacgtccctgctcagagcttctcaagtatctgaccacaaacgatgaccctcctcacaccaaacccacagaaaacaggaacagcagcagagacaaatgtgcttccaaaaagaagtcccatacacaaccgcagtcgcaacatgctcaagccaaaccaacaactttatctcttcctctgaccccagagtcaccaaatgaccccaagggttccccatttgagaacaagactattgagcgaaccttaagtgtggaactctctggaactgcaggcctaactcctcccacaactcctcctcataaagccaaccaagataaccctttcaaggcttcgccaaagctgaagccctcttgcaagaccgtggtgccaccgccaaccaagagggcccggtacagtgagtgttctggtacccaaggcagccactccaccaagaaagggcccgagcaatctgagttgtacgcacaactcagcaagtcctcagggctcagccgaggacacgaggaaaggaagactaaacggcccagtctccggctgtttggtgaccatgactactgtcagtcactcaattccaaaacggatatactcattaacatatcacaggagctccaagactctagacaactagacttcaaagatgcctcctgtgactggcaggggcacatctgttcttccacagattcaggccagtgctacctgagagagactttggaggccagcaagcaggtctctccttgcagcaccagaaaacagctccaagaccaggaaatccgagcggagctgaacaagcacttcggtcatccctgtcaagctgtgtttgacgacaaatcagacaagaccagtgaactaagggatggcgacttcagtaatgaacaattctccaaactacctgtgtttataaattcaggactagccatggatggcctatttgatgacagtgaagatgaaagtgataaactgagctacccttgggatggcacgcagccctattcattgttcgatgtgtcgccttcttgctcttcctttaactctccgtgtcgagactcagtgtcaccaccgaaatccttattttctcaaagaccccaaaggatgcgctctcgttcaagatccttttctcgacacaggtcgtgttcccgatcaccatattccaggtcaagatcaaggtccccaggcagtagatcctcttcaagatcctgttactactatgaatcaagccactacagacaccgcacacaccgcaattctcccttgtatgtgagatcacgttcaaggtcaccctacagccgtaggcccaggtacgacagctatgaagcctatgagcacgaaaggctcaagagggatgaataccgcaaagagcacgagaagcgggagtctgaaagggccaaacagagagagaggcagaagcagaaagcaattgaagagcgccgtgtgatttacgttggtaaaatcagacctgacacaacgcggacagaattgagagaccgctttgaagtttttggtgaaattgaggaatgcaccgtaaatctgcgggatgatggagacagctatggtttcatcacctaccgttacacctgtgacgctttcgctgctcttgagaatggatatactttacgcaggtcgaacgaaactgacttcgagctgtacttttgtggacggaagcaatttttcaagtctaactatgcagacctagataccaactcagacgattttgaccctgcttccaccaagagcaagtatgactctctggattttgatagtttactgaaggaagctcagagaagcttgcgcaggtaacgtgttcccaggctgaggaatgacagagagatggtcaatacctcatgggacagcgtgtcctttcccaagactcttgcaagtcatacttaggaatttctcctactttacactctctgtacaaaaataaaacaaaacaaaacaacaataacaacaacaacaacaacaataacaacaacaaccataccagaacaagaacaacggtttacatgaacacagctgctgaagaggcaagagacagaatgataatccagtaagcacacgtttattcacgggtgtcagctttgctttccctggaggctcttggtgacagtgtgtgtgcgtgtgtgtgtgtgggtgtgcgtgtgtgtatgtgtgtgtgtgtacttgtttggaaagtacatatgtacacatgtgaggacttgggggcacctgaacagaacgaacaagggcgaccccttcaaatggcagcatttccatgaagacacacttaaaacctacaacttcaaaatgttcgtattctatacaaaaggaaaataaataaatataaaaaaaaaaaaaaaaaaactcgagagatctatgaatcgtagatactgaaaaacccc

SEQ ID NO: 5 - protein, 797 aa, Mus musculus
MAWDMCSQDSVWSDIECAALVGEDQPLCPDLPELDLSELDVNDLDTDSFLGGLKWCSDQSEIISNQYNNEPANIFEKIDEENEANLLAVLTETLDSLPVDEDGLPSFDALTDGAVTTDNEASPSSMPDGTPPPQEAEEPSLLKKLLLAPANTQLSYNECSGLSTQNHAANHTHRIRTNPAIVKTENSWSNKAKSICQQQKPQRRPCSELLKYLTTNDDPPHTKPTENRNSSRDKCASKKKSHTQPQSQHAQAKPTTLSLPLTPESPNDPKGSPFENKTIERTLSVELSGTAGLTPPTTPPHKANQDNPFKASPKLKPSCKTVVPPPTKRARYSECSGTQGSHSTKKGPEQSELYAQLSKSSGLSRGHEERKTKRPSLRLFGDHDYCQSLNSKTDILINISQELQDSRQLDFKDASCDWQGHICSSTDSGQCYLRETLEASKQVSPCSTRKQLQDQEIRAELNKHFGHPCQAVFDDKSDKTSELRDGDFSNEQFSKLPVFINSGLAMDGLFDDSEDESDKLSYPWDGTQPYSLFDVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEAYEHERLKRDEYRKEHEKRESERAKQRERQKQKAIEERRVIYVGKIRPDTTRTELRDRFEVFGEIEECTVNLRDDGDSYGFITYRYTCDAFAALENGYTLRRSNETDFELYFCGRKQFFKSNYADLDTNSDDFDPASTKSKYDSLDFDSLLKEAQRSLRR

SEQ ID NO: 6 - DNA, 1893 bp, Mus musculus
gaattcggcacgaggcctgcatgagtgtgtgctgtgtgtcagagtggattggagttgaaaaagcttgactggcgtcattcgggagctggatggcttgggacatgtgcagccaagactctgtatggagtgacatagagtgtgctgctctggttggtgaggaccagcctctttgcccagatcttcctgaacttgacctttctgaacttgatgtgaatgacttggatacagacagctttctgggtggattgaagtggtgtagcgaccaatcggaaatcatatccaaccagtacaacaatgagcctgcgaacatatttgagaagatagatgaagagaatgaggcgaacttgctagcggtcctcacagagacactggacagtctccccgtggatgaagacggattgccctcatttgatgcactgacagatggagccgtgaccactgacaacgaggccagtccttcctccatgcctgacggcacccctccccctcaggaggcagaagagccgtctctacttaagaagctcttactggcaccagccaacactcagctcagctacaatgaatgcagcggtcttagcactcagaaccatgcagcaaaccacacccacaggatcagaacaaaccctgccattgttaagaccgagaattcatggagcaataaagcgaagagcatttgtcaacagcaaaagccacaaagacgtccctgctcagagcttctcaagtatctgaccacaaacgatgaccctcctcacaccaaacccacagaaaacaggaacagcagcagagacaaatgtgcttccaaaaagaagtcccatacacaaccgcagtcgcaacatgctcaagccaaaccaacaactttatctcttcctctgaccccagagtcaccaaatgaccccaagggttccccatttgagaacaagactattgagcgaaccttaagtgtggaactctctggaactgcagctccactagtgccaagggagcatccatgcatcattacatccaggtcgatattgaatgtcttcatgcaaagatgtctttctaatttataaatatgaacacatcacacaacttgtgttcattctattaaaggtgtaaaaactaatttgatttcaaaatagctgttgttagtaaagcaagatgagagaaaggagaatgttcttgtggcagaaggcatttaaatctattgcatatggagattttttttcagacactaccaacaggattttatgtctgaaatggaaatggaaaggcaatgtcagcctaacaaggtgatggcttgaaacacaagacatgaaggaactttgttagggaccaaaataactggtccccaattttatgtatatacatacatgttttggctatcactataaacatggtgaaagcaatggagctgttttataactgataaaaagatgaatagaacaaaataaccagctgtctttttactctcggaccactgggttctgcccatatttccttccattcacatatctttggttaccttgtttgaaatggggtagacatgcggttaatttggtttgttattatattatttgtttgaggatttcataaataagtgcaatatatttgcatcatttccaccccaacacctcccaaaaccacccatctcaaattcatttactctttttctataattgtttttgtcatatattacacacacacaaaggcgcatacacacacacgcacacacaggcacacacacacacacacacacacacacacacacacacacacactgagagttgccctaatttagggttgaccacttagggttcaggtctcatccctgaaaaatgaagaagaagaagaagaagaagaagaagaagaagaagaagaagaagaagaagaagaagaaaaaaaaaaaaaaaa

SEQ ID NO: 7 - protein, 320 aa, Mus musculus
MAWDMCSQDSVWSDIECAALVGEDQPLCPDLPELDLSELDVNDLDTDSFLGGLKWCSDQSEIISNQYNNEPANIFEKIDEENEANLLAVLTETLDSLPVDEDGLPSFDALTDGAVTTDNEASPSSMPDGTPPPQEAEEPSLLKKLLLAPANTQLSYNECSGLSTQNHAANHTHRIRTNPAIVKTENSWSNKAKSICQQQKPQRRPCSELLKYLTTNDDPPHTKPTENRNSSRDKCASKKKSHTQPQSQHAQAKPTTLSLPLTPESPNDPKGSPFENKTIERTLSVELSGTAAPLVPREHPCIITSRSILNVFMQRCLSNL

SEQ ID NO: 8 - DNA, 1744 bp, Mus musculus
  misc_feature 1543: n = A,T,C or G
gaattcggcacgaggtcagagtggattggagttgaaaaagcttgactggcgtcattcgggagctggatggcttgggacatgtgcagccaagactctgtatggagtgacatagagtgtgctgctctggttggtgaggaccagcctctttgcccagatcttcctgaacttgacctttctgaacttgatgtgaatgacttggatacagacagctttctgggtggattgaagtggtgtagcgaccaatcggaaatcatatccaaccagtacaacaatgagcctgcgaacatatttgagaagatagatgaagagaatgaggcaaacttgctagcggtcctcacagagacactggacagtctccccgtggatgaagacggattgccctcatttgatgcactgacagatggagccgtgaccactgacaacgaggccagtccttcctccatgcctgacggcacccctccccctcaggaggcagaagagccgtctctacttaagaagctcttactggcaccagccaacactcagctcagctacaatgaatgcagcggtcttagcactcagaaccatgcagcaaaccacacccacaggatcagaacaaaccctgccattgttaagaccgagaattcatggagcaataaagcgaagagcatttgtcaacagcaaaagccacaaagacgtccctgctcagagcttctcaagtatctgaccacaaacgatgaccctcctcacaccaaacccacagaaaacaggaacagcagcagagacaaatgtgcttccaaaaagaagtcccatacacaaccgcagtcgcaacatgctcaagccaaaccaacaactttatctcttcctctgaccccagagtcaccaaatgaccccaagggttccccatttgagaacaagactattgagcgaaccttaagtgtggaactctctggaactgcaggtgtaaaaactaatttgatttcaaaatagctgttgttagttaagcaagatgagagaaaggagaatgttcttgtggcagaaggcatttaaatctattgcatatggagattttttttcagacactaccaacaggattttatgtctgaaatggaaatggaaaggcaatgtcagcctaacaaggtgatggcttgaaacacaagacatgaaggaactttgttagggaccaaaataactggtccccaattttatgtatatacatacatgttttggctatcactataaacatggtgaaagcaatggagctgttttataactgataaaaagatgaatagaacaaaataaccagctgtctttttactctcggaccactgggttctgcccatatttccttccattcacatatctttggttaccttgtttgaaatggggtagacatgcggttaatttggtttgttattatattatttgtttgaggatttcataaataagtgcaatatatttgcatcatttccaccccaacacctcccaaaaccacccatctcaaattcatttactctttttctataattgtttttgtcatatattacacacacacaaaggcacntacacacacacgcacacacaggcacacacacacacacacacacacacacacacacacacacacactgagaattgccctaatttagggttgaccacttagggttcagtttttttccctggaaaatgggggggggggaaaaaaaaaaaaaaaaaaaaaaaaaaaaaaaaaaaaaaaaaaaaaaaaaaaaaaaaaaaaaaaaaa

SEQ ID NO: 9 - protein, 300 aa, Mus musculus
MAWDMCSQDSVWSDIECAALVGEDQPLCPDLPELDLSELDVNDLDTDSFLGGLKWCSDQSEIISNQYNNEPANIFEKIDEENEANLLAVLTETLDSLPVDEDGLPSFDALTDGAVTTDNEASPSSMPDGTPPPQEAEEPSLLKKLLLAPANTQLSYNECSGLSTQNHAANHTHRIRTNPAIVKTENSWSNKAKSICQQQKPQRRPCSELLKYLTTNDDPPHTKPTENRNSSRDKCASKKKSHTQPQSQHAQAKPTTLSLPLTPESPNDPKGSPFENKTIERTLSVELSGTAGVKTNLISK

SEQ ID NO: 10 - DNA, 3030 bp, Homo sapiens
atgcctcctgtgtatgcctctgagtatgtcttgccactccagggtggagggtccggggaggagcaactctatgctgactttccagaactcgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccatcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtggcgacccaacttttggcaagaagagctttgagcagaccttgacagtggagctctgtggcacagcaggtgagccagggggcttccactggcaggtgccttcaggaaaacacccgtgcatctctgagtttttcatcatgcatgggcaaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattga

SEQ ID NO: 11 - protein, 1009 aa, Homo sapiens
MPPVYASEYVLPLQGGGSGEEQLYADFPELDLSQLDASDFDSATCFGELQWCPENSETEPNQYSPDDSELFQIDSENEALLAELTKTLDDIPEDDVGLAAFPALDGGDALSCTSASPAPSSAPPSPAPEKPSAPAPEVDELSLLQKLLLATSYPTSSSDTQKEGTAWRQAGLRSKSQRPCVKADSTQDKKAPMMQSQSRSCTELHKHLTSAQCCLQDRGLQPPCLQSPRLPAKEDKEPGEDCPSPQPAPASPRDSLALGRADPGAPVSQEDMQAMVQLIRYMHTYCLPQRKLPPQTPEPLPKACSNPSQQVRSRPWSRHHSKASWAEFSILRELLAQDVLCDVSKPYRLATPVYASLTPRSRPRPPKDSQASPGRPSSVEEVRIAASPKSTGPRPSLRPLRLEVKREVRRPARLQQQEEEDEEEEEEEEEEEKEEEEEWGRKRPGRGLPWTKLGRKLESSVCPVRRSRRLNPELGPWLTFADEPLVPSEPQGALPSLCLAPKAYDVERELGSPTDEDSGQDQQLLRGPQIPALESPCESGDPTFGKKSFEQTLTVELCGTAGEPGGFHWQVPSGKHPCISEFFIMHGQGLTPPTTPPYKPTEEDPFKPDIKHSLGKEIALSLPSPEGLSLKATPGAAHKLPKKHPERSELLSHLRHATAQPASQAGQKRPFSCSFGDHDYCQVLRPEGVLQRKVLRSWEPSGVHLEDWPQQGAPWAEAQAPGREEDRSCDAGAPPKDSTLLRDHEIRASLTKHFGLLETALEEEDLASCKSPEYDTVFEDSSSSSGESSFLPEEEEEEGEEEEEDDEEEDSGVSPTCSDHCPYQSPPSKANRQLCSRSRSSSGSSPCHSWSPATRRNFRCESRGPCSDRTPSIRHARKRREKAIGEGRVVYIQNLSSDMSSRELKRRFEVFGEIEECEVLTRNRRGEKYGFITYRCSEHAALSLTKGAALRKRNEPSFQLSYGGLRHFCWPRYTDYDSNSEEALPASGKSKYEAMDFDSLLKEAQQSLH

SEQ ID NO: 12 - DNA, 3664 bp, Mus musculus
ctcgctccctcccccgggcgggctcggcgctgactccgccgcacgctgcagccgcggctggaagatggcggggaacgactgcggcgcgctgctggatgaagagctctcgtccttcttcctcaactatctctctgacacgcagggtggggactctggagaggaacagctgtgtgctgacttgccagagcttgacctctcccagctggacgccagtgactttgactcagccacgtgctttggggagctgcagtggtgcccggagacctcagagacagagcccagccagtacagccccgatgactccgagctcttccagattgacagtgagaatgaagctctcttggctgcgcttacgaagaccctggatgacatccccgaagacgatgtggggctggctgccttcccagaactggatgaaggcgacacaccatcctgcaccccagcctcacctgcccccttatctgcaccccccagccccaccctggagaggcttctgtccccagcgtctgacgtggacgagctttcactgctacagaagctcctcctggccacatcctccccaacagcaagctctgacgctctgaaggacggggccacctggtcccagaccagcctcagttccagaagtcagcggccttgtgtcaaggtggatggcacccaggataagaagacccccacactgcgggctcagagccggccttgtacggaactgcataagcacctcacttcggtgctgccctgtcccagagtgaaagcctgctccccaactccgcacccgagccctcggctcctctccaaagaggaggaggaggaggtgggggaggattgcccaagcccttggccgactccagcctcgccccaagactccctagcacaggacacggccagccccgacagtgcccagcctcccgaggaggatgtgagggccatggtacagctcattcgctacatgcatacctactgcctgcctcagaggaagctgccccaacgggccccagagccaatcccccaggcctgcagcagcctctccaggcaggttcaaccccgatcccggcatccccccaaagccttctggactgagttctctatcctaagggaacttctggcccaagatatcctctgtgatgttagcaagccctaccgcctggccatacctgtctatgcttccctcacacctcagtccaggcccaggccccccaaggacagtcaggcctcccctgcccactctgccatggcagaagaggtgagaatcactgcttcccccaagagcaccgggcctagacccagcctgcgtcctctgaggctggaggtgaaacgggatgttaacaagcctacaaggcaaaagcgggaggaagatgaggaggaggaggaggaagaagaagaagaggaagaagaaaaagaagaggaagaagaggagtggggcaggaagagaccaggtcgtggcctgccatggaccaacatagggaggaagatggacagctccgtgtgccccgtgcggcgctccaggagactgaatccagagctgggtccctggctgacattcactgatgagcccttaggtcctctgccctcgatgtgcctggatacagagacccacaacctggaggaagacctgggcagcctcacagacagtagtcaaggccggcagctcccccagggatcccagatccccgccctggaaagcccctgtgagagtgggtgcggagacacagatgaagatccaagctgcccacagcccacttccagagactcctccaggtgcctcatgctggccttgtcacaaagcgactctcttggcaagaagagctttgaggagtccctgacggtggagctttgcggcacggcaggactcacgccacccaccacacctccatacaagccaatggaggaggaccccttcaagccagacaccaagctcagcccaggccaagacacagctcccagccttccctcccccgaggctcttccgctcacagccaccccaggagcttcccacaagctgcccaagaggcacccagagcgaagcgagctcctgtcccatttgcagcatgccacaacccaaccagtctcacaggctggccagaagcgccccttctcctgctcctttggagaccacgactactgccaggtgctcaggccagaggctgccctgcagaggaaggtgctgcggtcctgggagccaatcggggtccaccttgaagacttggcccagcagggtgcccctctgccaacggaaacaaaggcccctaggagggaggcaaaccagaactgtgaccctacccacaaggacagcatgcagctaagagaccatgagatccgtgccagtctcacaaagcactttgggctgctggagactgctctggaaggtgaagacctggcgtcctgtaaaagcccggagtatgacaccgtatttgaggacagcagcagcagcagtggcgagagtagcttcctgcttgaggaggaggaggaagaggaggagggaggggaagaggacgatgaaggagaggactcaggggtcagccctccctgctctgatcactgcccctaccagagcccacccagtaaggccagtcggcagctctgctcccgaagccgctccagttccggctcctcgtcctgcagctcctggtcaccagccacccggaagaacttcagacgtgagagcagagggccctgttcagatggaaccccaagcgtccggcatgccaggaagcggcgggaaaaggccatcggtgaaggccgtgtggtatacattcgaaatctctccagtgacatgagctctcgggaactaaagaagcgctttgaggtgttcggtgagattgtagagtgccaggtgctgacgagaagtaaaagaggccagaagcacggttttatcagcttccggtgttcagagcacgctgccctgtccgtgaggaacggcgccaccctgagaaagcgcaatgagccctccttccacctgagctatggagggctccggcacttccgttggcccagatacactgactatgatcccacatctgaggagtcccttccctcatctgggaaaagcaagtacgaagccatggattttgacagcttactgaaagaggcccagcagagcctgcattgatatcagccttaaccttcgaggaatacctcaatacctcagacaaggcccttccaatatgtttacgttttcaaagaaaagagtatatgagaaggagagcgagcgagcgagcgagcgagcgagtgagcgtgagagatcacacaggagagagaaagacttgaatctgctgtcgtttcctttaaaaaaaaaaaaacgaaaaacaaaaacaaatcaatgtttacattgaacaaagctgcttccgtccgtctgtccgtccgtccgtccgtccgtgagttaccattctgatgatgttccactgccacgttagcgtcgtcctcgcttccagcggatcgtcctgggtgcgcctccaagtgctgtcagtcgtcctctgcccctcccacccgactgacttccttctgttagacttgagctgtgttcacataacatcttctgtctgtagagtgtgatgatgacattgttacttgtgaatagaatcaggagttagaaactcatttttaattgaagaaaaaaaaagtatatccttaaaaagaaaaaaaaaaaaacaaatgtaaaaaaaaa

SEQ ID NO: 13 - protein, 1014 aa, Mus musculus
MAGNDCGALLDEELSSFFLNYLSDTQGGDSGEEQLCADLPELDLSQLDASDFDSATCFGELQWCPETSETEPSQYSPDDSELFQIDSENEALLAALTKTLDDIPEDDVGLAAFPELDEGDTPSCTPASPAPLSAPPSPTLERLLSPASDVDELSLLQKLLLATSSPTASSDALKDGATWSQTSLSSRSQRPCVKVDGTQDKKTPTLRAQSRPCTELHKHLTSVLPCPRVKACSPTPHPSPRLLSKEEEEEVGEDCPSPWPTPASPQDSLAQDTASPDSAQPPEEDVRAMVQLIRYMHTYCLPQRKLPQRAPEPIPQACSSLSRQVQPRSRHPPKAFWTEFSILRELLAQDILCDVSKPYRLAIPVYASLTPQSRPRPPKDSQASPAHSAMAEEVRITASPKSTGPRPSLRPLRLEVKRDVNKPTRQKREEDEEEEEEEEEEEEEKEEEEEEWGRKRPGRGLPWTKLGRKMDSSVCPVRRSRRLNPELGPWLTFTDEPLGALPSMCLDTETHNLEEDLGSLTDSSQGRQLPQGSQIPALESPCESGCGDTDEDPSCPQPTSRDSSRCLMLALSQSDSLGKKSFEESLTVELCGTAGLTPPTTPPYKPMEEDPFKPDTKLSPGQDTAPSLPSPEALPLTATPGASHKLPKRHPERSELLSHLQHATTQPVSQAGQKRPFSCSFGDHDYCQVLRPEAALQRKVLRSWEPIGVHLEDLAQQGAPLPTETKAPRREANQNCDPTHKDSMQLRDHEIRASLTKHFGLLETALEGEDLASCKSPEYDTVFEDSSSSSGESSFLLEEEEEEEEGGEEDDEGEDSGVSPPCSDHCPYQSPPSKASRQLCSRSRSSSGSSSCSSWSPATRKNFRRESRGPCSDGTPSVRHARKRREKAIGEGRVVYIRNLSSDMSSRELKKRFEVFGEIVECQVLTRSKRGQKHGFITFRCSEHAALSVRNGATLRKRNEPSFHLSYGGLRHFRWPRYTDYDPTSEESLPSSGKSKYEAMDFDSLLKEAQQSLH

SEQ ID NO: 14 - DNA, 18 bp, Homo sapiens
  Domain
agtgacgtcaggagtttg

SEQ ID NO: 15 - DNA, 18 bp, Mus musculus
  Domain
agtgacgtcaggagtttg

SEQ ID NO: 16 - DNA, 18 bp, Artificial Sequence
  Domain
agtagatctaggagtttg

SEQ ID NO: 17 - protein, 18 aa, Artificial Sequence
  Domain
NKGNYTAAAAATANCYNN

SEQ ID NO: 18 - DNA, 18 bp, Homo sapiens
  Domain
ttacctaaatataatttg

SEQ ID NO: 19 - DNA, 18 bp, Mus musculus
gcatctaaatataattta

SEQ ID NO: 20 - DNA, 18 bp, Artificial Sequence
  Domain
gcatctccgcggaattta

(19) United States
(12) Patent Application Publication
(10) Pub. No.: US 2006/0035849 A1
(43) Pub. Date: Feb. 16, 2006

(54) Methods and composition for modulating type I muscle formation using pgc-1 alpha

(75) Inventors: Bruce M Spiegelman, Waban, MA (US); Jiandie Lin, Brookline, MA (US)

Correspondence Address: LAHIVE & COCKFIELD, LLP., 28 STATE STREET, BOSTON, MA, 02109, US

(73) Assignee: DanaFarber Cancer Institute, Inc, Boston, MA (US)

(21) Appl. No.: 10/514,531

(22) PCT Filed: Feb. 13, 2003
(86) PCT No.: PCT/US03/04792

§ 371 Date: Jun. 10, 2005

Related U.S. Application Data
(60) Provisional application No. 60/357,069, filed on Feb. 13, 2002.

(51) Int. Cl. A61K 48/00; C12N 15/861

(52) U.S. Cl. 514/44; 424/93.2; 435/456

(57) ABSTRACT

The invention provides novel methods and compositions for modulating type I muscle formation through modulation of PGC-1α activity or expression. Also provided are methods for identifying compounds that modulate type I muscle formation through modulation of PGC-1α activity or expression. Further provided are methods for treating disorders associated with type I and/or type II muscle formation, as well as transgenic animals expressing PGC-1α in muscle.

### RELATED APPLICATIONS

This application claims priority to U.S. provisional Application No. 60/357,069, filed on Feb. 13, 2002, incorporated herein in it's entirety by this reference.

### GOVERNMENT SUPPORT

Work described herein was supported under grant DK54477 awarded by the National Institutes of Health. The U.S. government may have certain rights in this invention.

### BACKGROUND OF THE INVENTION

The metabolic properties of muscle are profoundly influenced by exercise and disease. Long-term endurance exercise training or low frequency motor nerve stimulation promote the transition toward an oxidative metabolism with enhanced mitochondrial biogenesis characteristic of slow (type) skeletal muscle fibers. Conversely, disuse atrophy, exercise intolerance associated with congestive heart failure, and mitochondrial myopathies result in loss of type I oxidative skeletal muscle fibers, chronic fatigue, and increased glycolytic fibers.

Accordingly, there exists a need for additional therapeutic options which can modulate type I muscle formation to provide relief for symptoms of heart failure, disuse atrophy, mitochondrial myopathies, and systemic metabolic disorders.

### SUMMARY OF THE INVENTION

The present invention is based, at least in part, on the discovery that PGC-1α (also known as, and used interchangeably herein with, PGC-1), regulates type I (slow-twitch) muscle fiber differentiation and contributes to maintaining muscle cell determination. Accordingly, the present invention provides methods for modulating type I muscle formation comprising contacting a cell (i.e., a muscle cell such as a type I muscle cell or a type II muscle cell) with an agent that modulates PGC-1α expression or activity, such that type I muscle formation is modulated. In a preferred embodiment, PGC-1α expression or activity is increased, thereby increasing type I muscle formation.

In addition to PGC-1α, several additional factors involved in the signaling cascades underlying muscle fiber type determination have been identified, such as the calcium/calmodulin-dependent protein kinase IV (CaMKIV) and calcineurin A (CnA). It has been found that the PGC-1α promoter is regulated by both CaMKIV as well as CnA activity. CaMKIV activates PGC-1α almost entirely through a binding site for cAMP response element binding protein (CREB), which is found in the PGC-1α promoter. Moreover, a positive autoregulatory loop exists by which PGC-1α controls its own transcription through binding to and coactivation of myocyte enhancer factor 2 (MEF2) transcription factors, e.g., MEF2C and MEF2D, which are transcription factors that are targets of CaMKIV and CnA signaling and that bind directly to the PGC-1α promoter.

In one embodiment, the agent that modulates PGC-1α expression or activity is a PGC-1α nucleic acid molecule (i.e., a human PGC-1α nucleic acid molecule comprising the nucleic acid sequence of SEQ ID NO:1). In another embodiment, the PGC-1α nucleic acid molecule is contained within a vector. In yet another embodiment, the agent is a PGC-1α polypeptide (i.e., a human PGC-1α polypeptide comprising the amino acid sequence of SEQ ID NO:2). In a further embodiment, the agent is a small molecule.

The invention also provides methods for identifying compounds capable of modulating type I muscle formation comprising contacting a cell (i.e., a muscle cell such as a type I muscle cell or a type II muscle cell) with a compound, and determining whether PGC-1α expression or activity is modulated. In one embodiment, PGC-1α expression or activity is increased. In another embodiment, determining whether PGC-1α expression or activity is modulated is by measuring PGC-1α expression by Northern blotting. In another embodiment, determining whether PGC-1α expression or activity is modulated comprises determining whether expression of at least one of myoglobin, troponin I slow, troponin I fast, MCAD, COX II, COX IV, or cytochrome c is modulated. In still another embodiment, determining whether PGC-1α expression or activity is modulated comprises determining whether an MEF2 transcription factor is activated.

In another embodiment, the invention provides methods for identifying compounds capable of treating a disorder characterized by aberrant type I muscle formation (i.e., heart failure, disuse atrophy, mitochondrial myopathies, or systemic metabolic disorders) comprising identifying the ability of the compound to modulate the expression or activity of PGC-1α to thereby identify a compound capable of treating a disorder characterized by aberrant type I muscle formation. In a preferred embodiment, PGC-1α expression or activity is increased. In another embodiment, determining whether PGC-1α expression or activity is modulated is by measuring PGC-1α expression by Northern blotting. In yet another embodiment, determining whether PGC-1α expression or activity is modulated comprises determining whether expression of at least one of myoglobin, troponin I slow, troponin I fast, MCAD, COX II, COX IV, or cytochrome c is modulated. In another embodiment, the invention provides compounds identified by the methods of the invention. In still another embodiment, determining whether PGC-1α expression or activity is modulated comprises determining whether an MEF2 transcription factor is activated.

The invention further provides methods for treating subjects having disorders characterized by aberrant type I muscle formation (i.e., heart failure, disuse atrophy, mitochondrial myopathies, or systemic metabolic disorders), comprising administering to the subject an agent capable of modulating PGC-1α expression or activity, such that the disorder is treated. In one embodiment, PGC-1α expression or activity is increased. In another embodiment, type I muscle formation is increased. In yet another embodiment, the agent is a PGC-1α nucleic acid molecule (i.e., a human PGC-1α nucleic acid molecule comprising the nucleic acid sequence of SEQ ID NO:1). In a further embodiment, the PGC-1α nucleic acid molecule is contained within a vector. In yet a further embodiment, the agent is a small molecule.

The invention also provides transgenic non-human animals (i.e., mice, rats, monkeys, horses, dogs, turkeys, fish, cows, pigs, sheep, goats, frogs, chickens, etc.) comprising an exogenous PGC-1α nucleic acid molecule, wherein the exogenous PGC-1α nucleic acid molecule is expressed in the skeletal muscle of the non-human transgenic animal. In one embodiment, the exogenous PGC-1α nucleic acid molecule is operatively linked to a muscle-specific promoter (e.g., the muscle creatine kinase promoter, the dystrophin promoter, the myostatin promoter, the GDF-8 promoter, the UCP-3 promoter, the MyoD promoter, the MEF2 promoter, the myosin heavy chain promoter, the myosin light chain promoter, or a troponin promoter). In a preferred embodiment, the non-human animal is a mouse. In another preferred embodiment, the expression of at least one of myoglobin, troponin I slow, MCAD, COX II, COX IV, or cytochrome c is upregulated in the muscle cells of the non-human animal.

Other features and advantages of the invention will be apparent from the following detailed description and claims.

### BRIEF DESCRIPTION OF THE DRAWINGS

FIG. 1A-C depicts the induction of PGC-1α by CaMKIV via CREB. (A) CaMKIV-activation of PGC-1α can be abolished by the dominant negative ACREB. (B) The sequence of the CRE in the mouse PGC-1α promoter (SEQ ID NO:14) and the human promoter (SEQ ID NO:15). For a description of the CRE in the human PGC-1α promoter, see Herzig, S. et al. (2001) Nature 413, 179-183. The PGC-1α promoter with a mutation in the CRE site (ACRE) is also depicted (SEQ ID NO:16). (C) Site-directed mutagenesis of the CRE inhibits CaMKIV-mediated activation of PGC-1α.

FIG. 2A-B illustrates the coactivation of MEF2s on the PGC-1α promoter. (A) MEF2C and MEF2D activate the mouse PGC-1 promoter. (B) MEF2 activity is increased by CnA.

FIG. 3A-B depicts the activation of PGC-1 by MEF2C and MEF2D via a conserved binding site. (A) Identification of putative MEF2-binding sites in the human (SEQ ID NO:18) and mouse (SEQ ID NO:19) PGC-1α promoter. Both promoters were compared to the TRANSFAC transcription factor binding sites database (Quandt, K. et al (1995) Nucleic Acids Res 23, 4878-4884) and high-scoring hits to the matrix V$AMEF2.01 depicted (SEQ ID NO:17). In the TRANSFAC matrix, basepairs marked bold are of high information content and underlined basepairs denote the core sequence. Putative MEF2 binding sites in the PGC-1α promoters are bold and underlined. The PGC-1α promoter with a mutation in the MEF2 binding site (ΔMEF2) is also depicted (SEQ ID NO:20). (B) MEF2C and MEF2D activate a conserved MEF-response element in the PGC-1α promoter.

FIG. 4A-D depicts a model for the autoregulatory loop regulating PGC-1α in muscle fiber type determination. (B-D) Endogenous PGC-1α expression is increased in transgenic mice expressing ectopic PGC-1α in skeletal muscle.

### DETAILED DESCRIPTION OF THE INVENTION

The present invention is based, at least in part, on the discovery that PGC-1α can modulate type I (slow-twitch) muscle formation and mitochondrial biogenesis in muscle cells, as well as contribute to maintaining muscle cell determination. In particular, it has been found that PGC-1α can regulate type I muscle fiber differentiation. The present invention is further based, at least in part, on the discovery that transgenic animals expressing PGC-1α contain increased type I muscle fibers. Moreover, the muscles from these animals are more resistant to exercise-induced fatigue, a hallmark for slow-twitch muscle fibers and muscles following endurance training.

PGC-1α is a recently described coactivator of nuclear receptors and has been shown to play a major role in cellular respiration, adaptive thermogenesis, and gluconeogenesis in tissues such as brown fat and skeletal muscle (Puigserver, P. et al. (1998) Cell 92:829-839; Wu, Z. et al. (1999) Cell 98:115-124; Yoon J. C. et al. (2001) Nature 413 (6852):131-8. The discoveries of the instant invention implicate PGC-1α as a major regulator of type I muscle formation.

More specifically, it has been found that expression of PGC-1α in the muscles of transgenic mice induces dose-dependant expression of type I muscle specific genes (i.e., myoglobin and troponin I slow), as well as mitochondrial specific genes indicative of type I muscle specific mitochondrial biogenesis (i.e., MCAD, COX II, COX IV, and cytochrome c). PGC-1α expression in the muscles of the transgenic mice also induces a dose-dependant down-regulation of the expression of a type II muscle marker, troponin I fast. Induction of the type I specific genes (also referred to herein as “type I markers”), by PGC-1α in the muscles of the transgenic mice is seen in otherwise type II muscle fibers. Histological analysis indicates that the muscles of the transgenic mice have greater numbers of type I fibers than littermate controls, and the isolated muscle fibers are more resistant to exercise-induced fatigue. The discoveries of the instant invention thus identify PGC-1α as a major regulator of type I muscle differentiation.

In addition to PGC-1α, several additional factors involved in the signaling cascades underlying muscle fiber type determination have been identified, including CaMKIV and CnA. It has been found that the PGC-1α promoter is regulated by both CaMKIV as well as CnA activity. Exercise and subsequently elevated intracellular calcium levels result in an activation of both CaMKIV and CnA in skeletal muscle.

CaMKIV activates PGC-1α almost entirely through a binding site for CREB, which is found in the PGC-1α promoter (see Herzig, S., et al. (2001) Nature 413, 179-183 for a description of the cAMP responsive element (CRE) in the human PGC-1α promoter).

Moreover, there is a positive autoregulatory loop by which PGC-1α controls its own transcription through the binding to and coactivation of myocyte enhancer factor 2 (MEF2) transcription factors, e.g., MEF2C and MEF2D, which are transcription factors that are targets of CaMKIV and CnA signaling and that bind directly to the PGC-1α promoter. MEF2 transcription factors therefore can increase transcription of PGC-1α and this induction response is enhanced by the presence of PGC-1α. This positive autoregulatory loop helps to sustain high PGC-1α levels and thus promotes a stable expression of muscle fiber type I specific genes. It has also been found that ectopic expression of PGC-1α in the skeletal muscle of transgenic mice increased the levels of endogenous PGC-1α transcript.

These findings indicate that muscle fiber type determination may therefore maintain a quasi-stable state through the establishment of a regulatory loop involving PGC-1α and MEF2 proteins. In other words, once PGC-1α expression is triggered by, for example, exercise, PGC-1α expression levels are maintained without further muscle stimuli, thereby promoting a stable expression of muscle fiber type I specific genes. Knowledge of these mechanisms which control the regulation and maintenance of muscle fiber type determination allows for tissue-specific targeting of these and other factors in diseases with impaired muscle formation or general muscle wasting due to physical inactivity.

The instant invention therefore provides methods and compositions for modulating type I muscle formation using PGC-1α and modulators thereof. Accordingly, one aspect of the invention pertains to the use of PGC-1α molecules, referred to herein as PGC-1α nucleic acid and protein molecules, which comprise a family of molecules having certain conserved structural and functional features, and which play a role in or function in type I muscle formation associated activities. The term “family” when referring to the protein and nucleic acid molecules of the invention is intended to mean two or more proteins or nucleic acid molecules having a common structural domain and having sufficient amino acid or nucleotide sequence homology as defined herein. Such family members can be naturally occurring and can be from either the same or different species. For example, a family can contain a first protein of human origin, as well as other, distinct proteins of human origin or alternatively, can contain homologues of non-human origin. Members of a family may also have common functional characteristics.

Another aspect of the invention pertains to methods for treating a subject, having a disease or disorder characterized by (or associated with) aberrant or abnormal PGC-1α nucleic acid expression and/or PGC-1α protein activity. These methods include the step of administering a PGC-1α modulator to the subject such that treatment occurs. The language “aberrant or abnormal PGC-1α expression” refers to expression of a non-wild-type PGC-1α protein or a non-wild-type level of expression of a PGC-1α protein. Aberrant or abnormal PGC-1α protein activity refers to a non-wild-type PGC-1α protein activity or a non-wild-type level of PGC-1α protein activity. As the PGC-1α protein is involved in, for example, a pathway involving type I muscle formation, aberrant or abnormal PGC-1α protein activity or nucleic acid expression interferes with the normal expression of type I muscle specific genes, and/or type I muscle differentiation.

Non-limiting examples of disorders or diseases characterized by or associated with abnormal or aberrant PGC-1α protein activity or nucleic acid expression (also referred to herein as PGC-1α associated disorders or as type I muscle associated disorders) include cardiovascular disorders (i.e., heart failure), disuse atrophy, muscle wasting (i.e., that caused by disorders such as cancer, AIDS, or other infectious diseases), mitochondrial myopathies, systemic metabolic disorders (i.e., diabetes, insulin resistance, hypoglycemia, obesity, body weight disorders, cachexia, or anorexia). See Braunwald, E. et al. eds. Harrison's Principles of Internal Medicine, Eleventh Edition (McGraw-Hill Book Company, New York, 1987) pp. 1778-1797; Robbins, S. L. et al. Pathologic Basis of Disease, 3rd Edition (W.B. Saunders Company, Philadelphia, 1984) p. 972 for further descriptions of such disorders. The terms “treating” or “treatment,” as used herein, refer to reduction or alleviation of at least one adverse effect or symptom of a disorder or disease, i.e., a disorder or disease characterized by or associated with abnormal or aberrant PGC-1α protein activity or PGC-1α nucleic acid expression.

The terms “treating” or “treatment,” a used herein, further refers to increasing type I muscle formation in subjects without a type I muscle associated disorder, i.e., in subjects wherein increased type I muscle formation is desirable. For example, athletes, competitive racing animals, and other subjects wherein increased type I muscle formation would be desirable, may benefit from the methods of the present invention.

As used herein, a PGC-1α modulator is a molecule which can modulate PGC-1α nucleic acid expression and/or PGC-1α protein activity. For example, a PGC-1α modulator can modulate, i.e., upregulate (activate) or downregulate (suppress), PGC-1α nucleic acid expression. In another example, a PGC-1α modulator can modulate (i.e., stimulate or inhibit) PGC-1α protein activity. If it is desirable to treat a disorder or disease characterized by (or associated with) aberrant or abnormal (non-wild-type) PGC-1α nucleic acid expression and/or PGC-1α protein activity by inhibiting PGC-1α nucleic acid expression, a PGC-1α modulator can be an antisense molecule, i.e., a ribozyme, as described herein. Examples of antisense molecules which can be used to inhibit PGC-1α nucleic acid expression include antisense molecules which are complementary to a portion of the 5′ untranslated region of SEQ ID NO:1 or SEQ ID NO:4 which also includes the start codon and antisense molecules which are complementary to a portion of the 3′ untranslated region of SEQ ID NO:1 or SEQ ID NO:4.

A PGC-1α modulator which inhibits PGC-1α nucleic acid expression can also be a small molecule or other drug, i.e., a small molecule or drug identified using the screening assays described herein, which inhibits PGC-1α nucleic acid expression. A PGC-1α molecule of the invention can thus also be used as a target to screen molecules, i.e., which can modulate PGC-1α activity.

If it is desirable to treat a subject, by stimulating PGC-1α nucleic acid expression, PGC-1α modulator can be used, for example, a nucleic acid molecule encoding PGC-1α (i.e., a nucleic acid molecule comprising a nucleotide sequence homologous to the nucleotide sequence of SEQ ID NO:1 or SEQ ID NO:4), an active PGC-1α protein or portion thereof (i.e., a PGC-1α protein or portion thereof having an amino acid sequence which is homologous to the amino acid sequence of SEQ ID NO:2 or SEQ ID NO:5 or a portion thereof), or a small molecule or other drug, i.e., a small molecule (peptide) or drug identified using the screening assays described herein, which stimulates PGC-1α nucleic acid expression and/or PGC-1α protein activity.

Alternatively, if it is desirable to treat a disease or disorder characterized by (or associated with) aberrant or abnormal (non-wild-type) PGC-1α nucleic acid expression and/or PGC-1α protein activity by inhibiting PGC-1α protein activity, a PGC-1α modulator can be used, such as an anti-PGC-1α antibody or a small molecule or other drug, i.e., a small molecule or drug identified using the screening assays described herein, which inhibits PGC-1α protein activity. In a preferred embodiment, a PGC-1α modulator is a PGC-1α dominant negative.

If it is desirable to treat a disease or disorder characterized by (or associated with) aberrant or abnormal (non-wild-type) PGC-1α nucleic acid expression and/or PGC-1α protein activity by stimulating PGC-1α protein activity, a PGC-1α modulator can be an active PGC-1α protein or portion thereof (i.e., a PGC-1α protein or portion thereof having an amino acid sequence which is homologous to the amino acid sequence of SEQ ID NO:2 or SEQ ID NO:5 or a portion thereof) or a small molecule or other drug, i.e., a small molecule or drug identified using the screening assays described herein, which stimulates PGC-1α protein activity.

In addition, a subject having a type I muscle associated disorder (i.e., heart failure, disuse atrophy, a mitochondrial myopathy, or a systemic metabolic disorder), can be treated according to the present invention by administering to the subject a PGC-1α protein or portion thereof or a nucleic acid encoding a PGC-1α protein or portion thereof such that treatment occurs.

Other aspects of the invention pertain to methods for modulating a cell associated activity. These methods include contacting the cell with an agent (or a composition which includes an effective amount of an agent) which modulates PGC-1α protein activity or PGC-1α nucleic acid expression such that a cell associated activity is altered relative to a cell associated activity of the cell in the absence of the agent. As used herein, “a cell associated activity” refers to a normal or abnormal activity or function of a cell. Examples of cell associated activities include proliferation, migration, differentiation, production or secretion of molecules, such as proteins, cell survival, gluconeogenesis, and thermogenesis. In a preferred embodiment, the cell associated activity is type I muscle formation and the cell is a muscle cell. The term “altered” as used herein refers to a change, i.e., an increase or decrease, of a cell associated activity.

In one embodiment, the agent stimulates PGC-1α protein activity or PGC-1α nucleic acid expression. Examples of such stimulatory agents include an active PGC-1α protein, a nucleic acid molecule encoding PGC-1α that has been introduced into the cell, and a modulatory agent which stimulates PGC-1α protein activity or PGC-1α nucleic acid expression and which is identified using the drug screening assays described herein. In another embodiment, the agent inhibits PGC-1α protein activity or PGC-1α nucleic acid expression. Examples of such inhibitory agents include a nucleic acid molecule encoding a dominant negative PGC-1α protein, a dominant negative PGC-1α protein, an antisense PGC-1α nucleic acid molecule, an anti-PGC-1α antibody, and a modulatory agent which inhibits PGC-1α protein activity or PGC-1α nucleic acid expression and which is identified using the drug screening assays described herein. These modulatory methods can be performed in vitro (i.e., by culturing the cell with the agent) or, alternatively, in vivo (i.e., by administering the agent to a subject). In a preferred embodiment, the modulatory methods are performed in vivo, i.e., the cell is present within a subject, i.e., a mammal, i.e., a human, and the subject has a disorder or disease characterized by or associated with abnormal or aberrant PGC-1α protein activity or PGC-1α nucleic acid expression.

The methods of the present invention may therefore: 1) modulate type I muscle formation; 2) modulate the conversion of type II muscle fibers into type I muscle fibers; 3) modulate the response of muscle fibers to exercise induced fatigue; 4) treat diseases or disorders characterized by aberrant PGC-1α expression or activity, i.e., heart failure, disuse atrophy, mitochondrial myopathy, and/or systemic metabolic disease; 5) modulate the expression of myoglobin, troponin I slow, troponin I fast, MCAD, COX II, COX IV, and/or cytochrome c; and/or 6) modulate coactivation of MEF2 transcription factors, e.g., MEF2C and MEF2D.

A nucleic acid molecule, a protein, a PGC-1α modulator, a compound etc. used in the methods of treatment can be incorporated into an appropriate pharmaceutical composition described herein and administered to the subject through a route which allows the molecule, protein, modulator, or compound etc. to perform its intended function. Examples of routes of administration are also described herein.

The nucleotide sequence of the human PGC-1α cDNA and the predicted amino acid sequence of the human PGC-1α protein are shown in SEQ ID NOs:1 and 2, respectively. The human PGC-1α gene, which is approximately 3023 nucleotides in length, encodes a full length protein having a molecular weight of approximately 120 kD and which is approximately 798 amino acid residues in length. Further description of the human PGC-1α nucleic acid and polypeptide sequences can be found in PCT International Publication No. WO 00/32215, incorporated herein by reference.

The nucleotide sequence of the mouse PGC-1α cDNA and the predicted amino acid sequence of the mouse PGC-1α protein are shown in SEQ ID NOs:4 and 5, respectively. The mouse PGC-1α gene, which is approximately 3066 nucleotides in length, encodes a full length protein having a molecular weight of approximately 120 kD and which is approximately 797 amino acid residues in length. Further description of the mouse PGC-1α nucleic acid and polypeptide sequences can be found in PCT International Publication Nos. WO 00/32215 and WO 98/54220, U.S. Pat. No. 6,166,192, Puigserver, P. et al. (1998) Cell 92 (6):829-39, all of which are incorporated herein by reference.

PGC-1α family member proteins include several domains/motifs. These domains/motifs include: two putative tyrosine phosphorylation sites (amino acid residues 205-213 and 379-386 of SEQ ID NO:2, and amino acid residues 204-212 and 378-385 of SEQ ID NO:5), three putative cAMP phosphorylation sites (amino acid residues 239-242, 374-377, and 656-658 of SEQ ID NO:2, and 238-241, 373-376, and 655-658 of SEQ ID NO:5), a serine-arginine (SR) rich domain (amino acid residues 563-601 of SEQ ID NO:2, and 562-600 of SEQ ID NO:5), an RNA binding motif (amino acid residues 657-710 of SEQ ID NO:2, and 656-709 of SEQ ID NO:5), and an LXXLL motif (amino acid residues 144-148 of SEQ ID NO:2, and 142-146 of SEQ ID NO:5; SEQ ID NO:3) which mediates interaction with PPARγ, HNF-4α, and other nuclear receptors. As used herein, a tyrosine phosphorylation site is an amino acid sequence which includes at least one tyrosine residue which can be phosphorylated by a tyrosine protein kinase. Typically, a tyrosine phosphorylation site is characterized by a lysine or an arginine about seven residues to the N-terminal side of the phosphorylated tyrosine. An acidic residue (asparagine or glutamine) is often found at either three or four residues to the N-terminal side of the tyrosine (Patschinsky, T. et al. (1982) Proc. Natl. Acad. Sci. USA 79:973-977); Hunter, T. (1982) J. Biol. Chem. 257:4843-4848; Cooper, J. A. et al. (1984) J. Biol. Chem. 259:7835-7841). As used herein, a “cAMP phosphorylation site” is an amino acid sequence which includes a serine or threonine residue which can be phosphorylated by a cAMP-dependent protein kinase. Typically, the cAMP phosphorylation site is characterized by at least two consecutive basic residues to the N-terminal side of the serine or threonine (Fremisco, J. R. et al. (1980) J. Biol. Chem. 255:4240-4245; Glass, D. B. and Smith, S. B. (1983) J. Biol. Chem. 258:14797-14803; Glass, D. B. et al. (1986) J. Biol. Chem. 261:2987-2993). As used herein, a “serine-arginine rich domain” or an “SR rich domain” is an amino acid sequence which is rich in serine and arginine residues. Typically, SR rich domains are domains which interact with the CTD domain of RNA polymerase II or are involved in splicing functions. As used herein, an “RNA binding motif” is an amino acid sequence which can bind an RNA molecule or a single stranded DNA molecule. RNA binding motifs are described in Lodish, H., Darnell, J., and Baltimore, D. Molecular Cell Biology, 3rd ed. (W.H. Freeman and Company, New York, N.Y., 1995). As used herein, an “LXXLL motif” (SEQ ID NO:3) refers to a motif wherein L represents leucine and X can be any amino acid, and which mediates an interaction between a nuclear receptor and a coactivator (Heery et al. (1997) Nature 397:733-736; Torchia et al. (1997) Nature 387:677-684).

Various aspects of the invention are described in further detail in the following subsections:

I. Isolated Nucleic Acid Molecules

One aspect of the invention pertains to methods utilizing isolated nucleic acid molecules that encode PGC-1α or biologically active portions thereof, as well as nucleic acid fragments sufficient for use as hybridization probes to identify PGC-1α-encoding nucleic acid (i.e., PGC-1α mRNA). As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated PGC-1α nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., a brown adipocyte). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, i.e., a nucleic acid molecule having the nucleotide sequence of SEQ ID NO:1, SEQ ID NO:4 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1, SEQ ID NO:4 or a portion thereof (i.e., 400, 450, 500, or more nucleotides), can be isolated using standard molecular biology techniques and the sequence information provided herein. For example, a human PGC-1α cDNA can be isolated from a human liver, heart, kidney, or brain cell line (from Stratagene, LaJolla, Calif., or Clontech, Palo Alto, Calif.) using all or portion of SEQ ID NO:1 or SEQ ID NO:4 as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NO:1 or SEQ ID NO:4 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4 can be isolated by the polymerase chain reaction using oligonucleotide primers designed based upon the sequence of SEQ ID NO:1 or SEQ ID NO:4 or the homologous nucleotide sequence. For example, mRNA can be isolated from liver cells, heart cells, kidney cells, brain cells, or brown adipocytes (i.e., by the guanidinium-thiocyanate extraction procedure of Chirgwin et al. (1979) Biochemistry 18: 5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO:1 or SEQ ID NO:4 or to the homologous nucleotide sequence. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a PGC-1α nucleotide sequence can be prepared by standard synthetic techniques, i.e., using an automated DNA synthesizer.

In a preferred embodiment, an isolated nucleic acid molecule of the invention comprises the nucleotide sequence shown in SEQ ID NO:1 or SEQ ID NO:4 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4. The sequence of SEQ ID NO:4 corresponds to the mouse PGC-1α cDNA. This cDNA comprises sequences encoding the PGC-L a protein (i.e., “the coding region”, from nucleotides 92 to 2482), as well as 5′ untranslated sequences (nucleotides 1 to 91) and 3′ untranslated sequences (nucleotides 2483 to 3066). Alternatively, the nucleic acid molecule can comprise only the coding region of SEQ ID NO:4 (i.e., nucleotides 92 to 2482) or the homologous nucleotide sequence. The sequence of SEQ ID NO:1 corresponds to the human PGC-1α cDNA. This cDNA comprises sequences encoding the PGC-1α protein (i.e., “the coding region”, from nucleotides 89 to 2482), as well as 5′ untranslated sequences (nucleotides 1 to 88) and 3′ untranslated sequences (nucleotides 2513 to 3023). Alternatively, the nucleic acid molecule can comprise only the coding region of SEQ ID NO: (i.e., nucleotides 89 to 2482) or the homologous nucleotide sequence.

In another preferred embodiment, an isolated nucleic acid molecule of the invention comprises a nucleic acid molecule which is a complement of the nucleotide sequence shown in SEQ ID NO:1 or SEQ ID NO:4 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4. A nucleic acid molecule which is complementary to the nucleotide sequence shown in SEQ ID NO:1 or SEQ ID NO:4 or to a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4 is one which is sufficiently complementary to the nucleotide sequence shown in SEQ ID NO:1 or SEQ ID NO:4 or to the homologous sequence such that it can hybridize to the nucleotide sequence shown in SEQ ID NO:1 or SEQ ID NO:4 or to the homologous sequence, thereby forming a stable duplex.

In still another preferred embodiment, an isolated nucleic acid molecule of the invention comprises a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4 or a portion of this nucleotide sequence. In an additional preferred embodiment, an isolated nucleic acid molecule of the invention comprises a nucleotide sequence which hybridizes, i.e., hybridizes under stringent conditions, to the nucleotide sequence shown in SEQ ID NO:1 or SEQ ID NO:4 or to a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4.

Moreover, the nucleic acid molecule of the invention can comprise only a portion of the coding region of SEQ ID NO:1 or SEQ ID NO:4 or the coding region of a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or SEQ ID NO:4, for example a fragment which can be used as a probe or primer or a fragment encoding a biologically active portion of PGC-1α. The nucleotide sequence determined from the cloning of the PGC-1α gene from a mouse or human allows for the generation of probes and primers designed for use in identifying and/or cloning other PGC-1α family members, as well as PGC-1α homologues in other cell types, i.e. from other tissues, as well as PGC-1α homologues from other mammals such as rats or monkeys. The probe/primer typically comprises substantially purified oligonucleotide. The oligonucleotide typically comprises a region of nucleotide sequence that hybridizes under stringent conditions to at least about 12, preferably at least about 25, more preferably about 40, 50 or 75 consecutive nucleotides of SEQ ID NO:1 or SEQ ID NO:4 sense, an anti-sense sequence of SEQ ID NO:1 or SEQ ID NO:4, or naturally occurring mutants thereof. Primers based on the nucleotide sequence in SEQ ID NO:1 or SEQ ID NO:4 can be used in PCR reactions to clone PGC-1α homologues.

In an exemplary embodiment, a nucleic acid molecule of the present invention comprises a nucleotide sequence which is about 100, preferably 100-200, preferably 200-300, more preferably 300-400, and even more preferably 400-487 nucleotides in length and hybridizes under stringent hybridization conditions to a nucleic acid molecule of SEQ ID NO:1 or SEQ ID NO:4.

Probes based on the PGC-1α nucleotide sequences can be used to detect transcripts or genomic sequences encoding the same or homologous proteins. In preferred embodiments, the probe further comprises a label group attached thereto, i.e. the label group can be a radioisotope, a fluorescent compound, an enzyme, or an enzyme co-factor. Such probes can be used as a part of a diagnostic test kit for identifying cells or tissue which misexpress a PGC-1α protein, such as by measuring a level of a PGC-1α-encoding nucleic acid in a sample of cells from a subject i.e., detecting PGC-1α mRNA levels or determining whether a genomic PGC-1α gene has been mutated or deleted.

In one embodiment, the nucleic acid molecule of the invention encodes a protein or portion thereof which includes an amino acid sequence which is sufficiently homologous to an amino acid sequence of SEQ ID NO:2 or SEQ ID NO:5 such that the protein or portion thereof maintains one or more of the following biological activities: 1) it can modulate the expression of myoglobin, troponin I slow, troponin I fast, MCAD, COX II, COX IV, and/or cytochrome c; 2) it can modulate coactivation of MEF2 transcription factors; 3) it can modulate type I muscle formation; 4) it can modulate the conversion of type II muscle fibers into type I muscle fibers; 5) it can modulate the response of muscle fibers to exercise induced fatigue; and/or 6) it can treat diseases or disorders characterized by aberrant PGC-1α expression or activity, i.e., heart failure, disuse atrophy, mitochondrial myopathy, and/or systemic metabolic disease.

As used herein, the language “sufficiently homologous” refers to proteins or portions thereof which have amino acid sequences which include a minimum number of identical or equivalent (i.e., an amino acid residue which has a similar side chain as an amino acid residue in SEQ ID NO:2 or SEQ ID NO:5) amino acid residues to an amino acid sequence of SEQ ID NO:2 or SEQ ID NO:5 such that the protein or portion thereof maintains one or more of the following biological activities: 1) modulation of the expression of myoglobin, troponin I slow, troponin I fast, MCAD, COX II, COX IV, and/or cytochrome c; 2) modulate coactivation of MEF2 transcription factors; 3) modulation of type I muscle formation; 4) modulation of the conversion of type II muscle fibers into type I muscle fibers; 5) modulation of the response of muscle fibers to exercise induced fatigue; and/or 6) treatment of diseases or disorders characterized by aberrant PGC-1α expression or activity, i.e., heart failure, disuse atrophy, mitochondrial myopathy, and/or systemic metabolic disease.

In another embodiment, the protein is at least about 50%, preferably at least about 60%, more preferably at least about 70%, yet more preferably at least about 80%, still more preferably at least about 90%, and most preferably at least about 95% or more homologous to the entire amino acid sequence of SEQ ID NO:2 or SEQ ID NO:5.

Portions of proteins encoded by the PGC-1α nucleic acid molecule of the invention are preferably biologically active portions of the PGC-1α protein. As used herein, the term “biologically active portion of PGC-1α” is intended to include a portion, i.e., a domain/motif, of PGC-1α that has one or more of the following activities: 1) modulation of the expression of myoglobin, troponin I slow, troponin I fast, MCAD, COX II, COX IV, and/or cytochrome c; 2) modulate coactivation of MEF2 transcription factors; 3) modulation of type I muscle formation; 4) modulation of the conversion of type II muscle fibers into type I muscle fibers; 5) modulation of the response of muscle fibers to exercise induced fatigue; and/or 6) treatment of diseases or disorders characterized by aberrant PGC-1α expression or activity, i.e., heart failure, disuse atrophy, mitochondrial myopathy, and/or systemic metabolic disease. Standard binding assays, i.e., immunoprecipitations and yeast two-hybrid assays, as described herein, can be performed to determine the ability of a PGC-1α protein or a biologically active portion thereof to interact with (i.e., bind to) HNF-4α, FKHR, the PEPCK promoter, PPARγ, C/EBPα, NRF-1, or nuclear hormone receptors (i.e., known molecules which interact with PGC-1α). If a PGC-1α family member is found to interact with HNF-4α, FKHR, the PEPCK promoter, PPARγ, C/EBPα, NRF-1, or nuclear hormone receptors, then they are also likely to be modulators of the activity of HNF-4α, FKHR, the PEPCK promoter, PPARγ, C/EBPα, NRF-1, or nuclear hormone receptors.

To determine whether a PGC-1α family member of the present invention modulates myoglobin, troponin I slow, troponin I fast, MCAD, COX II, COX IV, and/or cytochrome c expression, in vitro transcriptional assays can be performed. To perform such an assay, the full length promoter/enhancer region of the gene of interest (i.e., myoglobin, troponin I slow, troponin I fast, MCAD, COX II, COX IV, and/or cytochrome c) can be linked to a reporter gene such as chloramphenicol acetyltransferase (CAT) or luciferase and introduced into host cells (i.e., liver cells such as Fao hepatoma cells, or COS cells). The same host cells can then be transfected with a nucleic acid molecule encoding the PGC-1α molecule. In some embodiments, nucleic acid molecules encoding HNF-4α, FKHR, NRF-1, and/or PPARγ/RXRα can also be transfected. The effect of the PGC-1α molecule can be measured by testing CAT or luciferase activity and comparing it to CAT or luciferase activity in cells which do not contain nucleic acid encoding the PGC-1α molecule. An increase or decrease in CAT or luciferase activity indicates a modulation of expression of the gene of interest. Because myoglobin, troponin I slow, MCAD, COX II, COX IV, and cytochrome c are known to be markers of mitochondrial biogenesis and/or type I muscle formation, and troponin I fast is known to be a marker of type II muscle, this assay can also measure the ability of the PGC-1α molecule to modulate type I muscle formation.

The above described assay for testing the ability of a PGC-1α molecule to modulate myoglobin, troponin I slow, troponin I fast, MCAD, COX II, COX IV, and cytochrome c expression can also be used to test the ability of the PGC-1α molecule to modulate type I muscle formation. If a PGC-1α molecule can modulate myoglobin, troponin I slow, troponin I fast, MCAD, COX II, COX IV, and/or cytochrome c expression, it can most likely modulate type I muscle formation. Alternatively, the ability of a PGC-1α molecule to modulate type I muscle formation can be measured by introducing a PGC-1α molecule into cells, i.e., a muscle cells, and measuring the amount of type I and type II muscle fibers that form.

In one embodiment, the biologically active portion of PGC-1α comprises at least one domain or motif. Examples of such domains/motifs include a tyrosine phosphorylation site, a cAMP phosphorylation site, a serine-arginine (SR) rich domain, an RNA binding motif, and an LXXLL (SEQ ID NO:3) motif which mediates interaction with HNF-4α and nuclear receptors. In one embodiment, the biologically active portion of the protein which includes the domain or motif can modulate differentiation of white adipocytes to brown adipocytes and/or thermogenesis in brown adipocytes or can modulate gluconeogenesis. In a preferred embodiment, the biologically active portion of the protein includes the domain or motif that can modulate mitochondrial biogenesis and/or type I muscle formation. These domains are described in detail herein. Additional nucleic acid fragments encoding biologically active portions of PGC-1α can be prepared by isolating a portion of SEQ ID NO:1 or SEQ ID NO:4 or a homologous nucleotide sequence, expressing the encoded portion of PGC-1α protein or peptide (i.e., by recombinant expression in vitro) and assessing the activity of the encoded portion of PGC-1α protein or peptide.

The invention further encompasses nucleic acid molecules that differ from the nucleotide sequence shown in SEQ ID NO:1 or SEQ ID NO:4 (and portions thereof) due to degeneracy of the genetic code and thus encode the same PGC-1α protein as that encoded by the nucleotide sequence shown in SEQ ID NO:1 or SEQ ID NO:4. In another embodiment, an isolated nucleic acid molecule of the invention has a nucleotide sequence encoding a protein having an amino acid sequence shown in SEQ ID NO:2 or SEQ ID NO:5 or a protein having an amino acid sequence which is at least about 50%, preferably at least about 60%, more preferably at least about 70%, yet more preferably at least about 80%, still more preferably at least about 90%, and most preferably at least about 95% or more homologous to the amino acid sequence of SEQ ID NO:2 or SEQ ID NO:5.

In addition to the mouse and human PGC-1α nucleotide sequences shown in SEQ ID NO:1 and SEQ ID NO:4, it will be appreciated by those skilled in the art that DNA sequence polymorphisms that lead to changes in the amino acid sequences of PGC-1α may exist within a population (i.e., a mammalian population, i.e., a human population). Such genetic polymorphism in the PGC-1α gene may exist among individuals within a population due to natural allelic variation. As used herein, the terms “gene” and “recombinant gene” refer to nucleic acid molecules comprising an open reading frame encoding a PGC-1α protein, preferably a mammalian, i.e., human, PGC-1α protein. Such natural allelic variations can typically result in 1-5% variance in the nucleotide sequence of the PGC-1α gene. Any and all such nucleotide variations and resulting amino acid polymorphisms in PGC-1α that are the result of natural allelic variation and that do not alter the functional activity of PGC-1α are intended to be within the scope of the invention. Moreover, nucleic acid molecules encoding PGC-1α proteins from other species, and thus which have a nucleotide sequence which differs from the human or mouse sequences of SEQ ID NO:1 and SEQ ID NO:4, are intended to be within the scope of the invention. Nucleic acid molecules corresponding to natural allelic variants and homologues of the mouse or human PGC-1α cDNAs of the invention can be isolated based on their homology to the mouse or human PGC-1α nucleic acid sequences disclosed herein using the mouse or human cDNA, or a portion thereof, as a hybridization probe according to standard hybridization techniques under stringent hybridization conditions (as described herein).

Moreover, nucleic acid molecules encoding other PGC-1α family members and thus which have a nucleotide sequence which differs from the PGC-1α sequences of SEQ ID NO:1 or SEQ ID NO:4 are intended to be within the scope of the invention. For example, the use of alternately-spliced isoforms of PGC-1α, referred to herein as PGC-1b and PGC-1c, or a PGC-1α homologue referred to herein as PGC-1β may be used in the methods of the invention. The nucleotide and amino acid sequences of mouse PGC-1b (SEQ ID NOs:6 and 7, respectively) are described in U.S. Provisional Patent Application No. 60/303,468, incorporated herein by reference. The nucleotide and amino acid sequences of mouse PGC-1c (SEQ ID NOs:8 and 9, respectively) are also described in U.S. Provisional Patent Application No. 60/303,468. The nucleotide and amino acid sequences of human (SEQ ID NOs:10 and 11, respectively) and mouse (SEQ ID NO:s:12 and 13, respectively) PGC-1β are described in U.S. Provisional Application No. 60/338,126 and in Lin, J. et al. (2002) J. Biol. Chem. 277 (3): 1645-8, incorporated herein by reference. The nucleotide and amino acid sequences of mouse PGC-1β are also described in GenBank Accession Nos. AF453324 and AAL47054, respectively.

Additionally, other PGC-1α family members, for example a PGC-3 cDNA, can be identified based on the nucleotide sequence of human PGC-1α or mouse PGC-1α. (It should be noted that a gene called PPARγ coactivator 2, or PGC-2, has already been described in the literature (Castillo, G. et al. (1999) EMBO J. 18 (13):3676-87). However, PGC-2 is both structurally and functionally unrelated to PGC-1α.) Moreover, nucleic acid molecules encoding PGC-1α proteins from different species, and thus which have a nucleotide sequence which differs from the PGC-1α sequences of SEQ ID NO:1 or SEQ ID NO:4 are intended to be within the scope of the invention. For example, rat or monkey PGC-1α cDNA can be identified based on the nucleotide sequence of a human PGC-1α.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1 or SEQ ID NO:4 or a nucleotide sequence which is about 60%, preferably at least about 70%, more preferably at least about 80%, still more preferably at least about 90%, and most preferably at least about 95% or more homologous to the nucleotide sequence of SEQ ID NO:1 or SEQ ID NO:4. In other embodiments, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1 or SEQ ID NO:4 corresponds to a naturally-occurring nucleic acid molecule. As used herein, a “naturally-occurring” nucleic acid molecule refers to an RNA or DNA molecule having a nucleotide sequence that occurs in nature (i.e., encodes a natural protein). In one embodiment, the nucleic acid encodes a natural human PGC-1α.

In addition to naturally-occurring allelic variants of the PGC-1α sequence that may exist in the population, the skilled artisan will further appreciate that changes can be introduced by mutation into the nucleotide sequence of SEQ ID NO:1 or SEQ ID NO:4, thereby leading to changes in the amino acid sequence of the encoded PGC-1α protein, without altering the functional ability of the PGC-1α protein. For example, nucleotide substitutions leading to amino acid substitutions at “non-essential” amino acid residues can be made in the sequence of SEQ ID NO:1 or SEQ ID NO:4. A “non-essential” amino acid residue is a residue that can be altered from the wild-type sequence of PGC-1α (i.e., the sequence of SEQ ID NO:2 or SEQ ID NO:5) without altering the activity of PGC-1α, whereas an “essential” amino acid residue is required for PGC-1α activity. For example, amino acid residues involved in the interaction of PGC-1α to binding partners or target molecules (i.e., those present in an LXXLL motif) are most likely essential residues of PGC-1α. Other amino acid residues, however, (i.e., those that are not conserved or only semi-conserved between mouse and human) may not be essential for activity and thus are likely to be amenable to alteration without altering PGC-1α activity. Furthermore, amino acid residues that are essential for PGC-1α functions related to thermogenesis, adipogenesis, or gluconeogenesis, but not essential for PGC-1α functions related to type I muscle formation, are likely to be amenable to alteration.

Accordingly, another aspect of the invention pertains to nucleic acid molecules encoding PGC-1α proteins that contain changes in amino acid residues that are not essential for PGC-1α activity. Such PGC-1α proteins differ in amino acid sequence from SEQ ID NO:2 or SEQ ID NO:5 yet retain at least one of the PGC-1α activities described herein. In one embodiment, the isolated nucleic acid molecule comprises a nucleotide sequence encoding a protein, wherein the protein comprises an amino acid sequence at least about 60% homologous to the amino acid sequence of SEQ ID NO:2 or SEQ ID NO:5 and is capable of modulating type I muscle formation. Preferably, the protein encoded by the nucleic acid molecule is at least about 70% homologous, preferably at least about 80-85% homologous, still more preferably at least about 90%, and most preferably at least about 95% homologous to the amino acid sequence of SEQ ID NO:2 or SEQ ID NO:5.

“Sequence identity or homology”, as used herein, refers to the sequence similarity between two polypeptide molecules or between two nucleic acid molecules. When a position in both of the two compared sequences is occupied by the same base or amino acid monomer subunit, i.e., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared ×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, i.e., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a PGC-1α protein homologous to the protein of SEQ ID NO:2 or SEQ ID NO:5 can be created by introducing one or more nucleotide substitutions, additions or deletions into the nucleotide sequence of SEQ ID NO:1 or SEQ ID NO:4 or a homologous nucleotide sequence such that one or more amino acid substitutions, additions or deletions are introduced into the encoded protein. Mutations can be introduced into SEQ ID NO:1 or SEQ ID NO:4 or the homologous nucleotide sequence by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (i.e., lysine, arginine, histidine), acidic side chains (i.e., aspartic acid, glutamic acid), uncharged polar side chains (i.e., glycine, asparagine, glutamine, serine, threonine, tyrosine, cysteine), nonpolar side chains (i.e., alanine, valine, leucine, isoleucine, proline, phenylalanine, methionine, tryptophan), beta-branched side chains (i.e., threonine, valine, isoleucine) and aromatic side chains (i.e., tyrosine, phenylalanine, tryptophan, histidine). Thus, a predicted nonessential amino acid residue in PGC-1α is preferably replaced with another amino acid residue from the same side chain family. Alternatively, in another embodiment, mutations can be introduced randomly along all or part of a PGC-1α coding sequence, such as by saturation mutagenesis, and the resultant mutants can be screened for a PGC-1α activity described herein to identify mutants that retain PGC-1α activity. Following mutagenesis of SEQ ID NO:1 or SEQ ID NO:4, the encoded protein can be expressed recombinantly (as described herein) and the activity of the protein can be determined using, for example, assays described herein.

In addition to the nucleic acid molecules encoding PGC-1α proteins described above, another aspect of the invention pertains to isolated nucleic acid molecules which are antisense thereto. An “antisense” nucleic acid comprises a nucleotide sequence which is complementary to a “sense” nucleic acid encoding a protein, i.e., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire PGC-1α coding strand, or to only a portion thereof. In one embodiment, an antisense nucleic acid molecule is antisense to a “coding region” of the coding strand of a nucleotide sequence encoding PGC-1α. The term “coding region” refers to the region of the nucleotide sequence comprising codons which are translated into amino acid residues (i.e., the entire coding region of SEQ ID NO:4 comprises nucleotides 92 to 2482, the entire coding region of SEQ ID NO:1 comprises nucleotides 89 to 2482). In another embodiment, the antisense nucleic acid molecule is antisense to a “noncoding region” of the coding strand of a nucleotide sequence encoding PGC-1α. The term “noncoding region” refers to 5′ and 3′ sequences which flank the coding region that are not translated into amino acids (i.e., also referred to as 5′ and 3′ untranslated regions).

Given the coding strand sequences encoding PGC-1α disclosed herein (i.e., SEQ ID NO:1 and SEQ ID NO:4), antisense nucleic acids of the invention can be designed according to the rules of Watson and Crick base pairing. The antisense nucleic acid molecule can be complementary to the entire coding region of PGC-1α mRNA, but more preferably is an oligonucleotide which is antisense to only a portion of the coding or noncoding region of PGC-1α mRNA. For example, the antisense oligonucleotide can be complementary to the region surrounding the translation start site of PGC-1α mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (i.e., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, i.e., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a PGC-1α protein to thereby inhibit expression of the protein, i.e., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, i.e., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (i.e., hammerhead ribozymes (described in Haseloff and Gerlach (1988) Nature 334:585-591)) can be used to catalytically cleave PGC-1α mRNA transcripts to thereby inhibit translation of PGC-1α mRNA. A ribozyme having specificity for a PGC-1α-encoding nucleic acid can be designed based upon the nucleotide sequence of a PGC-1α cDNA disclosed herein (i.e., SEQ ID NO:1 or SEQ ID NO:4). For example, a derivative of a Tetrahymena L-19 IVS RNA can be constructed in which the nucleotide sequence of the active site is complementary to the nucleotide sequence to be cleaved in a PGC-1α-encoding mRNA. See, i.e., Cech et al. U.S. Pat. No. 4,987,071 and Cech et al. U.S. Pat. No. 5,116,742. Alternatively, PGC-1α mRNA can be used to select a catalytic RNA having a specific ribonuclease activity from a pool of RNA molecules. See, i.e., Bartel, D. and Szostak, J. W. (1993) Science 261:1411-1418.

Alternatively, PGC-1α gene expression can be inhibited by targeting nucleotide sequences complementary to the regulatory region of the PGC-1α (i.e., the PGC-1α promoter and/or enhancers) to form triple helical structures that prevent transcription of the PGC-1α gene in target cells. See generally, Helene, C. (1991) Anticancer Drug Des. 6 (6):569-84; Helene, C. et al. (1992) Ann. N.Y. Acad. Sci 660:27-36; and Maher, L. J. (1992) Bioassays 14 (12):807-15.

II. Recombinant Expression Vectors and Host Cells

Another aspect of the invention pertains to the use of vectors, preferably expression vectors, containing a nucleic acid encoding PGC-1α (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (i.e., bacterial vectors having a bacterial origin of replication and episomal mammalian vectors). Other vectors (i.e., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (i.e., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (i.e., in an in vitro transcription/translation system or in a host cell when the vector is introduced into the host cell). The term “regulatory sequence” is intended to includes promoters, enhancers and other expression control elements (i.e., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (i.e., tissue-specific regulatory sequences). In a preferred embodiment, a muscle specific promoter is used to direct expression of the nucleotide sequence in muscle (e.g., in a type I muscle cell or in a type II muscle cell). Muscle specific promoters include, without limitation, the muscle creatine kinase promoter, the dystrophin promoter, the myostatin promoter, the GDF-8 promoter, the UCP-3 promoter, the MyoD promoter, the MEF2 the promoter, the myosin heavy chain promoter, the myosin light chain promoter, and a troponin promoter. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (i.e., PGC-1α proteins, mutant forms of PGC-1α, fusion proteins, etc.).

The recombinant expression vectors of the invention can be designed for expression of PGC-1α in prokaryotic or eukaryotic cells. For example, PGC-1α can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the PGC-1α is cloned into a pGEX expression vector to create a vector encoding a fusion protein comprising, from the N-terminus to the C-terminus, GST-thrombin cleavage site-PGC-1α. The fusion protein can be purified by affinity chromatography using glutathione-agarose resin. Recombinant PGC-1α unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the PGC-1α expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, PGC-1α can be expressed in insect cells using baculovirus expression vectors. Baculovirus vectors available for expression of proteins in cultured insect cells (i.e., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (i.e., tissue-specific regulatory elements are used to express the nucleic acid). Tissue-specific regulatory elements are known in the art. Non-limiting examples of suitable tissue-specific promoters include the muscle specific casein kinase promoter, the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (i.e., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (i.e., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to PGC-1α mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1 (1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, PGC-1α protein can be expressed in bacterial cells such as E. coli, insect cells, yeast or mammalian cells (such as muscle cells, Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (i.e., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (i.e., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding PGC-1α or can be introduced on a separate vector. Cells stably transfected with the introduced nucleic acid can be identified by drug selection (i.e., cells that have incorporated the selectable marker gene will survive, while the other cells die).

A host cell of the invention, such as a prokaryotic or eukaryotic host cell in culture, can be used to produce (i.e., express) PGC-1α protein. Accordingly, the invention further provides methods for producing PGC-1α protein using the host cells of the invention. In one embodiment, the method comprises culturing the host cell of invention (into which a recombinant expression vector encoding PGC-1α has been introduced) in a suitable medium until PGC-1α is produced. In another embodiment, the method further comprises isolating PGC-1α from the medium or the host cell.

III. Transgenic Animals

The host cells of the invention can also be used to produce nonhuman transgenic animals. The nonhuman transgenic animals (i.e., mice, rats, monkeys, horses, dogs, turkeys, fish, cows, pigs, sheep, goats, frogs, or chickens) can be used, for example, in screening assays designed to identify agents or compounds, i.e., drugs, pharmaceuticals, etc., which are involved with type I muscle formation and/or capable of ameliorating detrimental symptoms of type I muscle associated disorders.

For example, in one embodiment, a host cell of the invention is a fertilized oocyte or an embryonic stem cell into which PGC-1α-coding sequences have been introduced. Such host cells can then be used to create non-human transgenic animals in which exogenous PGC-1α sequences have been introduced into their genome or homologous recombinant animals in which endogenous PGC-1α sequences have been altered. Such animals are useful for studying the function and/or activity of PGC-1α and for identifying and/or evaluating modulators of PGC-1α activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous PGC-1α gene has been altered by homologous recombination between the endogenous gene and an exogenous DNA molecule introduced into a cell of the animal, i.e., an embryonic cell of the animal, prior to development of the animal.

A transgenic animal of the invention can be created by introducing PGC-1α-encoding nucleic acid into the male pronuclei of a fertilized oocyte, i.e., by microinjection, retroviral infection, and allowing the oocyte to develop in a pseudopregnant female foster animal. The human PGC-1α cDNA sequence can be introduced as a transgene into the genome of a nonhuman animal. Alternatively, a nonhuman homologue of the human PGC-1α gene (SEQ ID NO:1), such as a mouse PGC-1α gene (SEQ ID NO:4), can used as a transgene. Intronic sequences and polyadenylation signals can also be included in the transgene to increase the efficiency of expression of the transgene. A tissue-specific regulatory sequence(s) can be operably linked to the PGC-1α transgene to direct expression of PGC-1α protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the PGC-1α transgene in its genome and/or expression of PGC-1α mRNA in tissues or cells of the animals. A transgenic founder animal can then be used to breed additional animals carrying the transgene. Moreover, transgenic animals carrying a transgene encoding PGC-1α can further be bred to other transgenic animals carrying other transgenes.

To create a homologous recombinant animal, a vector is prepared which contains at least a portion of a PGC-1α gene into which a deletion, addition or substitution has been introduced to thereby alter, i.e., functionally disrupt, the PGC-1α gene. The PGC-1α gene can be a human gene (i.e., from a human genomic clone isolated from a human genomic library screened with the cDNA of SEQ ID NO:1), but more preferably, is a nonhuman homologue of a human PGC-1α gene. For example, a mouse PGC-1α gene can be used to construct a homologous recombination vector suitable for altering an endogenous PGC-1α gene in the mouse genome. In a preferred embodiment, the vector is designed such that, upon homologous recombination, the endogenous PGC-1α gene is functionally disrupted (i.e., no longer encodes a functional protein; also referred to as a “knock out” vector). Alternatively, the vector can be designed such that, upon homologous recombination, the endogenous PGC-1α gene is mutated or otherwise altered but still encodes functional protein (i.e., the upstream regulatory region can be altered to thereby alter the expression of the endogenous PGC-1α protein). In the homologous recombination vector, the altered portion of the PGC-1α gene is flanked at its 5′ and 3′ ends by additional nucleic acid of the PGC-1α gene to allow for homologous recombination to occur between the exogenous PGC-1α gene carried by the vector and an endogenous PGC-1α gene in an embryonic stem cell. The additional flanking PGC-L a nucleic acid is of sufficient length for successful homologous recombination with the endogenous gene. Typically, several kilobases of flanking DNA (both at the 5′ and 3′ ends) are included in the vector (see i.e., Thomas, K. R. and Capecchi, M. R. (1987) Cell 51:503 for a description of homologous recombination vectors). The vector is introduced into an embryonic stem cell line (i.e., by electroporation) and cells in which the introduced PGC-1α gene has homologously recombined with the endogenous PGC-1α gene are selected (see i.e., Li, E. et al. (1992) Cell 69:915). The selected cells are then injected into a blastocyst of an animal (i.e., a mouse) to form aggregation chimeras (see i.e., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijistra et al.; and WO 93/04169 by Berns et al.

In one embodiment of the invention, transgenic animals are created using a vector containing a muscle specific promoter operatively linked to a PGC-1α nucleic acid molecule. Non-limiting examples of muscle specific promoters include muscle creatine kinase, dystrophin, myostatin (Gonzalez-Cadavid, N. F. et al. (1998) Proc. Natl. Acad. Sci. USA 95 (25):1493843), GDF-8 (PCT International Publication No. WO 00/04051), UCP-3, MyoD, MEF2, myosin heavy chain, myosin light chain, and various forms of troponin.

In another preferred embodiment of the invention, transgenic mouse strains were generated which express PGC-1α from the muscle creatine kinase promoter. The PGC-1α cDNA sequence was placed under the control of a muscle-specific promoter (muscle creatine kinase (MCK) promoter). Transgenic mice were generated using DNA microinjection and screened by PCR. Four independent founder lines were obtained (line #29, line #23, line #26, and line #31) and mated with wild type mice to obtain progeny for use in experiments.

Lines #23 and #31 show strong PGC-1α mRNA expression, line #26 shows low PGC-1α expression, while line #29 shows little PGC-1α expression. These mice show a PGC-1α dose-dependant increase in the expression of type I specific marker gene expression in the muscle, a dose-dependant decrease in the expression of type II specific marker gene expression in the muscle, and an increase in type I muscle fiber content, as determined by metachromatic and anti-myosin histological analysis. The transgenic mice have a greatly increased amount of dark-colored (type I) muscle throughout their entire bodies, including the hind-limb muscles. More specifically, the gastrocnemius muscle (normally a type II muscle) is the same dark color in the transgenic mice as the soleus (type I) muscle. The muscle fibers isolated from the transgenic mice also are more resistant to exercise-induced fatigue, a hallmark for slow-twitch muscle fibers and muscles following endurance training.

In another embodiment, transgenic nonhuman animals can be produced which contain selected systems which allow for regulated expression of the transgene. One example of such a system is the cre/loxP recombinase system of bacteriophage P1. For a description of the cre/loxP recombinase system, see, i.e., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, i.e., by mating two transgenic animals, one containing a transgene encoding a selected protein and the other containing a transgene encoding a recombinase.

Clones of the nonhuman transgenic animals described herein can also be produced according to the methods described in Wilmut, I. et al. (1997) Nature 385:810-813 and PCT International Publication Nos. WO 97/07668 and WO 97/07669. In brief, a cell, i.e., a somatic cell, from the transgenic animal can be isolated and induced to exit the growth cycle and enter Go phase. The quiescent cell can then be fused, i.e., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, i.e., the somatic cell, is isolated.

IV. Isolated PGC-1α Proteins and Anti-PGC-1α Antibodies

Another aspect of the invention pertains to the use of isolated PGC-1α proteins, and biologically active portions thereof, as well as peptide fragments suitable for use as immunogens to raise anti-PGC-1α antibodies. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of PGC-1α protein in which the protein is separated from cellular components of the cells in which it is naturally or recombinantly produced. In one embodiment, the language “substantially free of cellular material” includes preparations of PGC-1α protein having less than about 30% (by dry weight) of non-PGC-1α protein (also referred to herein as a “contaminating protein”), more preferably less than about 20% of non-PGC-1α protein, still more preferably less than about 10% of non-PGC-1α protein, and most preferably less than about 5% non-PGC-1α protein. When the PGC-1α protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of PGC-1α protein in which the protein is separated from chemical precursors or other chemicals which are involved in the synthesis of the protein. In one embodiment, the language “substantially free of chemical precursors or other chemicals” includes preparations of PGC-1α protein having less than about 30% (by dry weight) of chemical precursors or non-PGC-1α chemicals, more preferably less than about 20% chemical precursors or non-PGC-1α chemicals, still more preferably less than about 10% chemical precursors or non-PGC-1α chemicals, and most preferably less than about 5% chemical precursors or non-PGC-1α chemicals. In preferred embodiments, isolated proteins or biologically active portions thereof lack contaminating proteins from the same animal from which the PGC-1α protein is derived. Typically, such proteins are produced by recombinant expression of, for example, a human PGC-1α protein in a nonhuman cell.

An isolated PGC-1α protein or a portion thereof of the invention has one or more of the following biological activities: 1) modulation of the expression of myoglobin, troponin I slow, troponin I fast, MCAD, COX II, COX IV, and/or cytochrome c; 2) modulate coactivation of MEF2 transcription factors; 3) modulation of type I muscle formation; 4) modulation of the conversion of type II muscle fibers into type I muscle fibers; 5) modulation of the response of muscle fibers to exercise induced fatigue; and/or 6) treatment of diseases or disorders characterized by aberrant PGC-1α expression or activity, i.e., heart failure, disuse atrophy, mitochondrial myopathy, and/or systemic metabolic disease. In preferred embodiments, the protein or portion thereof comprises an amino acid sequence which is sufficiently homologous to an amino acid sequence of SEQ ID NO:2 or SEQ ID NO:5 such that the protein or portion thereof maintains the ability to modulate gluconeogenesis. The portion of the protein is preferably a biologically active portion as described herein. In another preferred embodiment, the PGC-1α protein (i.e., amino acid residues 1-797 or amino acid residues 1-798) has an amino acid sequence shown in SEQ ID NO:2 or SEQ ID NO:5, respectively, or an amino acid sequence which is at least about 50%, preferably at least about 60%, more preferably at least about 70%, yet more preferably at least about 80%, still more preferably at least about 90%, and most preferably at least about 95% or more homologous to the amino acid sequence shown in SEQ ID NO:2 or SEQ ID NO:5. In yet another preferred embodiment, the PGC-1α protein has an amino acid sequence which is encoded by a nucleotide sequence which hybridizes, i.e., hybridizes under stringent conditions, to the nucleotide sequence of SEQ ID NO:1 or SEQ ID NO:4 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1, SEQ ID NO:4. The preferred PGC-1α proteins of the present invention also preferably possess at least one of the PGC-1α biological activities described herein. For example, a preferred PGC-1α protein of the present invention includes an amino acid sequence encoded by a nucleotide sequence which hybridizes, i.e., hybridizes under stringent conditions, to the nucleotide sequence of SEQ ID NO:1 or SEQ ID NO:4 and which can modulate gluconeogenesis.

In other embodiments, the PGC-1α protein is substantially homologous to the amino acid sequence of SEQ ID NO:2 or SEQ ID NO:5 and retains the functional activity of the protein of SEQ ID NO:2 or SEQ ID NO:5 yet differs in amino acid sequence due to natural allelic variation or mutagenesis, as described in detail in subsection I above. Accordingly, in another embodiment, the PGC-1α protein is a protein which comprises an amino acid sequence which is at least about 50%, preferably at least about 60%, more preferably at least about 70%, yet more preferably at least about 80%, still more preferably at least about 90%, and most preferably at least about 95% or more homologous to the amino acid sequence of SEQ ID NO:2, SEQ ID NO:5.

Biologically active portions of the PGC-1α protein include peptides comprising amino acid sequences derived from the amino acid sequence of the PGC-1α protein, i.e., the amino acid sequence shown in SEQ ID NO:2 or SEQ ID NO:5 or the amino acid sequence of a protein homologous to the PGC-1α protein, which include fewer amino acids than the full length PGC-1α protein or the full length protein which is homologous to the PGC-1α protein, and exhibit at least one activity of the PGC-1α protein. Typically, biologically active portions (peptides, i.e., peptides which are, for example, 5, 10, 15, 20, 30, 35, 36, 37, 38, 39, 40, 50, 100 or more amino acids in length) comprise a domain or motif, i.e., a tyrosine phosphorylation site, a cAMP phosphorylation site, a serine-arginine (SR) rich domain, and/or an RNA binding motif, with at least one activity of the PGC-1α protein. In a preferred embodiment, the biologically active portion of the protein which includes one or more the domains/motifs described herein can modulate type I muscle formation, mitochondrial biogenesis, as well as differentiation of adipocytes and/or thermogenesis in brown adipocytes, and/or gluconeogenesis. Moreover, other biologically active portions, in which other regions of the protein are deleted, can be prepared by recombinant techniques and evaluated for one or more of the activities described herein. Preferably, the biologically active portions of the PGC-1α protein include one or more selected domains/motifs or portions thereof having biological activity.

PGC-1α proteins are preferably produced by recombinant DNA techniques. For example, a nucleic acid molecule encoding the protein is cloned into an expression vector (as described above), the expression vector is introduced into a host cell (as described above) and the PGC-1α protein is expressed in the host cell. The PGC-1α protein can then be isolated from the cells by an appropriate purification scheme using standard protein purification techniques. Alternative to recombinant expression, a PGC-1α protein, polypeptide, or peptide can be synthesized chemically using standard peptide synthesis techniques. Moreover, native PGC-1α protein can be isolated from cells (i.e., brown adipocytes), for example using an anti-PGC-1α antibody (described further below).

The invention also provides PGC-1α chimeric or fusion proteins. As used herein, a PGC-1α “chimeric protein” or “fusion protein” comprises a PGC-1α polypeptide operatively linked to a non-PGC-1α polypeptide. A “PGC-1α polypeptide” refers to a polypeptide having an amino acid sequence corresponding to PGC-1α, whereas a “non-PGC-1α polypeptide” refers to a polypeptide having an amino acid sequence corresponding to a protein which is not substantially homologous to the PGC-1α protein, i.e., a protein which is different from the PGC-1α protein and which is derived from the same or a different organism. Within the fusion protein, the term “operatively linked” is intended to indicate that the PGC-1α polypeptide and the non-PGC-1α polypeptide are fused in-frame to each other. The non-PGC-1α polypeptide can be fused to the N-terminus or C-terminus of the PGC-1α polypeptide. For example, in one embodiment the fusion protein is a GST-PGC-1α fusion protein in which the PGC-1α sequences are fused to the C-terminus of the GST sequences. Such fusion proteins can facilitate the purification of recombinant PGC-1α. In another embodiment, the fusion protein is a PGC-1α protein containing a heterologous signal sequence at its N-terminus. In certain host cells (i.e., mammalian host cells), expression and/or secretion of PGC-1α can be increased through use of a heterologous signal sequence.

Preferably, a PGC-1α chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (i.e., a GST polypeptide). A PGC-1α-encoding nucleic acid can be cloned into such an expression vector such that the fusion moiety is linked in-frame to the PGC-1α protein.

The present invention also pertains to homologues of the PGC-1α proteins which function as either a PGC-1α agonist (mimetic) or a PGC-1α antagonist. In a preferred embodiment, the PGC-1α agonists and antagonists stimulate or inhibit, respectively, a subset of the biological activities of the naturally occurring form of the PGC-1α protein. Thus, specific biological effects can be elicited by treatment with a homologue of limited function. In one embodiment, treatment of a subject with a homologue having a subset of the biological activities of the naturally occurring form of the protein has fewer side effects in a subject relative to treatment with the naturally occurring form of the PGC-1α protein.

Homologues of the PGC-1α protein can be generated by mutagenesis, i.e., discrete point mutation or truncation of the PGC-1α protein. As used herein, the term “homologue” refers to a variant form of the PGC-1α protein which acts as an agonist or antagonist of the activity of the PGC-1α protein. An agonist of the PGC-1α protein can retain substantially the same, or a subset, of the biological activities of the PGC-1α protein. An antagonist of the PGC-1α protein can inhibit one or more of the activities of the naturally occurring form of the PGC-1α protein, by, for example, competitively binding to a downstream or upstream member of the PGC-1α cascade which includes the PGC-1α protein. Thus, the mammalian PGC-1α protein and homologues thereof of the present invention can be, for example, either positive or negative regulators of adipocyte differentiation and/or thermogenesis in brown adipocytes.

In an alternative embodiment, homologues of the PGC-1α protein can be identified by screening combinatorial libraries of mutants, i.e., truncation mutants, of the PGC-1α protein for PGC-1α protein agonist or antagonist activity. In one embodiment, a variegated library of PGC-1α variants is generated by combinatorial mutagenesis at the nucleic acid level and is encoded by a variegated gene library. A variegated library of PGC-1α variants can be produced by, for example, enzymatically ligating a mixture of synthetic oligonucleotides into gene sequences such that a degenerate set of potential PGC-1α sequences is expressible as individual polypeptides, or alternatively, as a set of larger fusion proteins (i.e., for phage display) containing the set of PGC-1α sequences therein. There are a variety of methods which can be used to produce libraries of potential PGC-1α homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PGC-1α sequences. Methods for synthesizing degenerate oligonucleotides are known in the art (see, i.e., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the PGC-1α protein coding can be used to generate a variegated population of PGC-1α fragments for screening and subsequent selection of homologues of a PGC-1α protein. In one embodiment, a library of coding sequence fragments can be generated by treating a double stranded PCR fragment of a PGC-1α coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the PGC-1α protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PGC-1α homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify PGC-1α homologues (Arkin and Youvan (1992) Proc. Natl. Acad. Sci. USA 89:7811-7815; Delagrave et al. (1993) Protein Eng. 6 (3):327-331).

An isolated PGC-1α protein, or a portion or fragment thereof, can be used as an immunogen to generate antibodies that bind PGC-1α using standard techniques for polyclonal and monoclonal antibody preparation. The full-length PGC-1α protein can be used or, alternatively, the invention provides antigenic peptide fragments of PGC-1α for use as immunogens. The antigenic peptide of PGC-1α comprises at least 8 amino acid residues of the amino acid sequence shown in SEQ ID NO:2, SEQ ID NO:5 or a homologous amino acid sequence as described herein and encompasses an epitope of PGC-1α such that an antibody raised against the peptide forms a specific immune complex with PGC-1α. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of PGC-1α that are located on the surface of the protein, i.e., hydrophilic regions.

A PGC-1α immunogen typically is used to prepare antibodies by immunizing a suitable subject, (i.e., rabbit, goat, mouse or other mammal) with the immunogen. An appropriate immunogenic preparation can contain, for example, recombinantly expressed PGC-1α protein or a chemically synthesized PGC-1α peptide. The preparation can further include an adjuvant, such as Freund's complete or incomplete adjuvant, or similar immunostimulatory agent. Immunization of a suitable subject with an immunogenic PGC-1α preparation induces a polyclonal anti-PGC-1α antibody response.

Accordingly, another aspect of the invention pertains to anti-PGC-1α antibodies. The term “antibody” as used herein refers to immunoglobulin molecules and immunologically active portions of immunoglobulin molecules, i.e., molecules that contain an antigen binding site which specifically binds (immunoreacts with) an antigen, such as PGC-1α. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind PGC-1α. The term “monoclonal antibody” or “monoclonal antibody composition”, as used herein, refers to a population of antibody molecules that contain only one species of an antigen binding site capable of immunoreacting with a particular epitope of PGC-1α. A monoclonal antibody composition thus typically displays a single binding affinity for a particular PGC-1α protein with which it immunoreacts.

Polyclonal anti-PGC-1α antibodies can be prepared as described above by immunizing a suitable subject with a PGC-1α immunogen. The anti-PGC-1α antibody titer in the immunized subject can be monitored over time by standard techniques, such as with an enzyme linked immunosorbent assay (ELISA) using immobilized PGC-1α. If desired, the antibody molecules directed against PGC-1α can be isolated from the mammal (i.e., from the blood) and further purified by well known techniques, such as protein A chromatography to obtain the IgG fraction. At an appropriate time after immunization, i.e., when the anti-PGC-1α antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a PGC-1α immunogen as described above, and the culture supernatants of the resulting hybridoma cells are screened to identify a hybridoma producing a monoclonal antibody that binds PGC-1α.

Any of the many well known protocols used for fusing lymphocytes and immortalized cell lines can be applied for the purpose of generating an anti-PGC-1α monoclonal antibody (see, i.e., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (i.e., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, i.e., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind PGC-1α, i.e., using a standard ELISA assay.

Alternative to preparing monoclonal antibody-secreting hybridomas, a monoclonal anti-PGC-1α antibody can be identified and isolated by screening a recombinant combinatorial immunoglobulin library (i.e., an antibody phage display library) with PGC-1α to thereby isolate immunoglobulin library members that bind PGC-1α. Kits for generating and screening phage display libraries are commercially available (i.e., the Pharmacia Recombinant Phage Antibody System, Catalog No. 27-9400-01; and the Stratagene SurfZAP™ Phage Display Kit, Catalog No. 2406.12). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. Nature (1990) 348:552-554.

Additionally, recombinant anti-PGC-1α antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-PGC-1α antibody (i.e., monoclonal antibody) can be used to isolate PGC-1α by standard techniques, such as affinity chromatography or immunoprecipitation. An anti-PGC-1α antibody can facilitate the purification of natural PGC-1α from cells and of recombinantly produced PGC-1α expressed in host cells. Moreover, an anti-PGC-1α antibody can be used to detect PGC-1α protein (i.e., in a cellular lysate or cell supernatant) in order to evaluate the abundance and pattern of expression of the PGC-1α protein. Anti-PGC-1α antibodies can be used diagnostically to monitor protein levels in tissue as part of a clinical testing procedure, i.e., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

V. Pharmaceutical Compositions

The PGC-1α nucleic acid molecules, PGC-1α proteins, PGC-1α modulators, and anti-PGC-1α antibodies (also referred to herein as “active compounds”) of the invention can be incorporated into pharmaceutical compositions suitable for administration to a subject, i.e., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, i.e., intravenous, intradermal, subcutaneous, oral (i.e., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., a PGC-1α protein or anti-PGC-1α antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, i.e., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (i.e., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

VI. Gene Therapy

In a preferred embodiment, the nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see i.e., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, i.e. retroviral vectors, the pharmaceutical preparation can include one or more cells which produce the gene delivery system.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, particularly into humans. Adenovirus preferentially targets the liver when administered systemically (greater than 90+%; (Antinozzi et al. (1999) Annu. Rev. Nutr. 19:511-544) for reasons that may have to do with the expression of viral receptors or the lack of vascular barriers in the liver. Alternatively they can be used for introducing exogenous genes ex vivo into liver cells in culture. These vectors provide efficient delivery of genes into liver cells, and the transferred nucleic acids are stably integrated into the chromosomal DNA of the host cell.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) is replaced by a gene of interest rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (i.e. lactose to convert the env protein to an asialoglycoprotein), as well as by generating fusion proteins (i.e. single-chain antibody/env fusion proteins). Thus, in a specific embodiment of the invention, viral particles containing a nucleic acid molecule containing a gene of interest operably linked to appropriate regulatory elements, are modified for example according to the methods described above, such that they can specifically target subsets of liver cells. For example, the viral particle can be coated with antibodies to surface molecule that are specific to certain types of liver cells. This method is particularly useful when only specific subsets of liver cells are desired to be transfected.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (i.e., Ad2, Ad3, Ad7 etc.) are well known to those skilled in the art. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (i.e., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, i.e., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Yet another viral vector system useful for delivery of a nucleic acid molecule comprising a gene of interest is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics Microbiol. Immunol. (1992) 158:97-129). Adeno-associated viruses exhibit a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as few as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into T cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Other viral vector systems that may have application in gene therapy have been derived from herpes virus, vaccinia virus, and several RNA viruses.

Still another viral vector system useful for delivery of a nucleic acid molecule comprising a gene of interest include the Herpes simplex virus type 1 (HSV-1) amplicon vectors for transfer of a gene into muscle (Wang, Y. et al. (2002) Hum. Gene. Ther. 13 (2):261-273);

Other methods relating to the use of viral vectors in gene therapy can be found in, i.e., Kay, M. A. (1997) Chest 111 (6 Supp.): 138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Invetig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

The pharmaceutical compositions can be included in a container, pack, or dispenser together with instructions for administration.

VII. Uses and Methods of the Invention

The nucleic acid molecules, polypeptides, polypeptide homologues, modulators, and antibodies described herein can be used in methods of treatment as well as drug screening assays. A PGC-1α protein of the invention has one or more of the activities described herein and can thus be used to, for example, modulate mitochondrial biogenesis and/or type I muscle formation. The isolated nucleic acid molecules of the invention can be used to express PGC-1α protein (i.e., via a recombinant expression vector in a host cell in gene therapy applications), to detect PGC-1α mRNA (i.e., in a biological sample) or a genetic lesion in a PGC-1α gene, and to modulate PGC-1α activity, as described further below. In addition, the PGC-1α proteins can be used to screen drugs or compounds which modulate PGC-1α protein activity as well as to treat disorders characterized by insufficient excessive production of PGC-1α protein or production of PGC-1α protein forms which have increased or decreased activity compared to wild type PGC-1α. Moreover, the anti-PGC-1α antibodies of the invention can be used to detect and isolate PGC-1α protein and modulate PGC-1α protein activity.

The invention provides a method (also referred to herein as a “screening assay”) for identifying modulators, i.e., candidate or test compounds or agents (i.e., peptides, peptidomimetics, small molecules or other drugs) which bind to PGC-1α proteins, have a stimulatory or inhibitory effect on, for example, PGC-1α expression or PGC-1α activity, or have a stimulatory or inhibitory effect on, for example, the expression or activity of a PGC-1α target molecule.

In one embodiment, the invention provides assays for screening candidate or test compounds which are target molecules of a PGC-1α protein or polypeptide or biologically active portion thereof. In another embodiment, the invention provides assays for screening candidate or test compounds which bind to or modulate the activity of a PGC-1α protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (i.e., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses a PGC-1α protein or biologically active portion thereof is contacted with a test compound and the ability of the test compound to modulate PGC-1α activity is determined. Determining the ability of the test compound to modulate PGC-1α activity can be accomplished by monitoring, for example, PEPCK, glucose-6-phosphatase, and/or fructose-1,6-bisphosphatase expression; and/or glucose release into the culture medium in a cell which expresses PGC-1α. The cell, for example, can be of mammalian origin, i.e., an Fao hepatoma cell.

The ability of the test compound to modulate PGC-1α binding to a target molecule can also be determined. Determining the ability of the test compound to modulate PGC-1α binding to a target molecule can be accomplished, for example, by coupling the PGC-1α target molecule with a radioisotope or enzymatic label such that binding of the PGC-1α target molecule to PGC-1α can be determined by detecting the labeled PGC-1α target molecule in a complex. Alternatively, PGC-1α could be coupled with a radioisotope or enzymatic label to monitor the ability of a test compound to modulate PGC-1α binding to a PGC-1α target molecule in a complex. Determining the ability of the test compound to bind PGC-1α can be accomplished, for example, by coupling the compound with a radioisotope or enzymatic label such that binding of the compound to PGC-1α can be determined by detecting the labeled PGC-1α compound in a complex. For example, compounds (i.e., PGC-1α target molecules) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound or target molecule to interact with PGC-1α without the labeling of any of the interactants. For example, a microphysiometer can be used to detect the interaction of a compound with PGC-1α without the labeling of either the compound or the PGC-1α. McConnell, H. M. et al. (1992) Science 257:1906-1912. As used herein, a “microphysiometer” (i.e., Cytosensor) is an analytical instrument that measures the rate at which a cell acidifies its environment using a light-addressable potentiometric sensor (LAPS). Changes in this acidification rate can be used as an indicator of the interaction between a compound and PGC-1α.

In another embodiment, an assay is a cell-based assay comprising contacting a cell expressing a PGC-1α target molecule with a test compound and determining the ability of the test compound to modulate (i.e. stimulate or inhibit) the activity of the PGC-1α target molecule. Determining the ability of the test compound to modulate the activity of a PGC-1α target molecule can be accomplished, for example, by determining the ability of a PGC-1α protein to bind to or interact with the PGC-1α target molecule, or by determining the ability of a PGC-1α protein to induce expression from a reporter construct.

Determining the ability of the PGC-1α protein, or a biologically active fragment thereof, to bind to or interact with a PGC-1α target molecule can be accomplished by one of the methods described above for determining direct binding. In a preferred embodiment, determining the ability of the PGC-1α protein to bind to or interact with a PGC-1α target molecule can be accomplished by determining the activity of the target molecule. For example, the activity of the target molecule can be determined by detecting induction of a cellular response (i.e., expression of type I muscle specific genes or mitochondrial specific genes), detecting catalytic/enzymatic activity of the target molecule upon an appropriate substrate, detecting the induction of a reporter gene (comprising a target-responsive regulatory element operatively linked to a nucleic acid encoding a detectable marker, i.e., luciferase), or detecting a target-regulated cellular response (i.e., differentiation into type I muscle or resistance to response to induced fatigue).

In yet another embodiment, an assay of the present invention is a cell-free assay in which a PGC-1α protein or biologically active portion thereof is contacted with a test compound and the ability of the test compound to bind to the PGC-1α protein or biologically active portion thereof is determined. Preferred biologically active portions of the PGC-1α proteins to be used in assays of the present invention include fragments which participate in interactions with target molecules. Binding of the test compound to the PGC-1α protein can be determined either directly or indirectly as described above. In a preferred embodiment, the assay includes contacting the PGC-1α protein or biologically active portion thereof with a known compound which binds PGC-1α to form an assay mixture, contacting the assay mixture with a test compound, and determining the ability of the test compound to interact with a PGC-1α protein, wherein determining the ability of the test compound to interact with a PGC-1α protein comprises determining the ability of the test compound to preferentially bind to PGC-1α or biologically active portion thereof as compared to the known compound.

In another embodiment, the assay is a cell-free assay in which a PGC-1α protein or biologically active portion thereof is contacted with a test compound and the ability of the test compound to modulate (i.e., stimulate or inhibit) the activity of the PGC-1α protein or biologically active portion thereof is determined. Determining the ability of the test compound to modulate the activity of a PGC-1α protein can be accomplished, for example, by determining the ability of the PGC-1α protein to bind to a PGC-1α target molecule by one of the methods described above for determining direct binding. Determining the ability of the PGC-1α protein to bind to a PGC-1α target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (i.e., BIAcore). Changes in the optical phenomenon of surface plasmon resonance (SPR) can be used as an indication of real-time reactions between biological molecules.

In an alternative embodiment, determining the ability of the test compound to modulate the activity of a PGC-1α protein can be accomplished by determining the ability of the PGC-1α protein to further modulate the activity of a downstream effector of a PGC-1α target molecule. For example, the activity of the effector molecule on an appropriate target can be determined or the binding of the effector to an appropriate target can be determined as previously described.

In yet another embodiment, the cell-free assay involves contacting a PGC-1α protein or biologically active portion thereof with a known compound which binds the PGC-1α protein (i.e., PPAR(, HNF-4(, FKHR, or the PEPCK promoter) to form an assay mixture, contacting the assay mixture with a test compound, and determining the ability of the test compound to interact with the PGC-1α protein, wherein determining the ability of the test compound to interact with the PGC-1α protein comprises determining the ability of the PGC-1α protein to preferentially bind to or modulate the activity of a PGC-1α target molecule.

In more than one embodiment of the above assay methods of the present invention, it may be desirable to immobilize either PGC-1α or its target molecule to facilitate separation of complexed from uncomplexed forms of one or both of the proteins, as well as to accommodate automation of the assay. Binding of a test compound to a PGC-1α protein, or interaction of a PGC-1α protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-5-transferase/PGC-1α fusion proteins or glutathione-5-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or PGC-1α protein, and the mixture incubated under conditions conducive to complex formation (i.e., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of PGC-1α binding or activity determined using standard techniques.

Other techniques for immobilizing proteins on matrices can also be used in the screening assays of the invention. For example, either a PGC-1α protein or a PGC-1α target molecule can be immobilized utilizing conjugation of biotin and streptavidin. Biotinylated PGC-1α protein or target molecules can be prepared from biotin-NHS(N-hydroxy-succinimide) using techniques known in the art (i.e., biotinylation kit, Pierce Chemicals, Rockford, Ill.), and immobilized in the wells of streptavidin-coated 96 well plates (Pierce Chemical). Alternatively, antibodies reactive with PGC-1α protein or target molecules but which do not interfere with binding of the PGC-1α protein to its target molecule can be derivatized to the wells of the plate, and unbound target or PGC-1α protein trapped in the wells by antibody conjugation. Methods for detecting such complexes, in addition to those described above for the GST-immobilized complexes, include immunodetection of complexes using antibodies reactive with the PGC-1α protein or target molecule, as well as enzyme-linked assays which rely on detecting an enzymatic activity associated with the PGC-1α protein or target molecule.

In another embodiment, modulators of PGC-1α expression are identified in a method wherein a cell is contacted with a candidate compound and the expression of PGC-1α mRNA or protein in the cell is determined. The level of expression of PGC-1α mRNA or protein in the presence of the candidate compound is compared to the level of expression of PGC-1α mRNA or protein in the absence of the candidate compound. The candidate compound can then be identified as a modulator of PGC-1α expression based on this comparison. For example, when expression of PGC-1α mRNA or protein is greater (statistically significantly greater) in the presence of the candidate compound than in its absence, the candidate compound is identified as a stimulator of PGC-1α mRNA or protein expression. Alternatively, when expression of PGC-1α mRNA or protein is less (statistically significantly less) in the presence of the candidate compound than in its absence, the candidate compound is identified as an inhibitor of PGC-1α mRNA or protein expression. The level of PGC-1α mRNA or protein expression in the cells can be determined by methods described herein for detecting PGC-1α mRNA or protein.

In yet another aspect of the invention, the PGC-1α proteins can be used as “bait proteins” in a two-hybrid assay or three-hybrid assay (see, i.e., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with PGC-1α (“PGC-1α-binding proteins” or “PGC-1α-bp”) and are involved in PGC-1α activity. Such PGC-1α-binding proteins are also likely to be involved in the propagation of signals by the PGC-1α proteins or PGC-1α targets as, for example, downstream elements of a PGC-1α-mediated signaling pathway. Alternatively, such PGC-1α-binding proteins may be PGC-1α inhibitors.

The two-hybrid system is based on the modular nature of most transcription factors, which consist of separable DNA-binding and activation domains. Briefly, the assay utilizes two different DNA constructs. In one construct, the gene that codes for a PGC-1α protein is fused to a gene encoding the DNA binding domain of a known transcription factor (i.e., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PGC-1α-dependent complex, the DNA-binding and activation domains of the transcription factor are brought into close proximity. This proximity allows transcription of a reporter gene (i.e., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the PGC-1α protein.

In another aspect, the invention pertains to a combination of two or more of the assays described herein. For example, a modulating agent can be identified using a cell-based or a cell-free assay, and the ability of the agent to modulate the activity of a PGC-1α protein can be confirmed in vivo, i.e., in an animal such as a mouse transgenic for PGC-1α, particularly wherein the PGC-1α is expressed in the muscle. Compounds can also be tested in wild-type mice for the ability to increase type I muscle fiber formation. Other animals useful in the methods of the invention include those with heart failure, disuse atrophy, mitochondrial myopathies, and/or systemic metabolic diseases.

This invention further pertains to novel agents identified by the above-described screening assays. Accordingly, it is within the scope of this invention to further use an agent identified as described herein in an appropriate animal model. For example, an agent identified as described herein (i.e., a PGC-1α modulating agent, an antisense PGC-1α nucleic acid molecule, a PGC-1α-specific antibody, or a PGC-1α binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

In yet another embodiment, the invention provides a method for identifying a compound (i.e., a screening assay) capable of use in the treatment of a disorder characterized by (or associated with) aberrant or abnormal PGC-1α nucleic acid expression or PGC-1α polypeptide activity. This method typically includes the step of assaying the ability of the compound or agent to modulate the expression of the PGC-1α nucleic acid or the activity of the PGC-1α protein thereby identifying a compound for treating a disorder characterized by aberrant or abnormal PGC-1α nucleic acid expression or PGC-1α polypeptide activity. Disorders characterized by aberrant or abnormal PGC-1α nucleic acid expression or PGC-1α protein activity are described herein. Methods for assaying the ability of the compound or agent to modulate the expression of the PGC-1α nucleic acid or activity of the PGC-1α protein are typically cell-based assays. For example, cells which are sensitive to ligands which transduce signals via a pathway involving PGC-1α can be induced to overexpress a PGC-1α protein in the presence and absence of a candidate compound. Candidate compounds which produce a statistically significant change in PGC-1α-dependent responses (either stimulation or inhibition) can be identified. In one embodiment, expression of the PGC-1α nucleic acid or activity of a PGC-1α protein is modulated in cells and the effects of candidate compounds on the readout of interest (such as rate of cell proliferation or differentiation) are measured. For example, the expression of genes which are up- or down-regulated in response to a PGC-1α protein-dependent signal cascade (i.e., myoglobin, troponin I slow, troponin I fast, MCAD, COX II, COX IV, and/or cytochrome c) can be assayed. In preferred embodiments, the regulatory regions of such genes, i.e., the 5′ flanking promoter and enhancer regions, are operably linked to a detectable marker (such as luciferase) which encodes a gene product that can be readily detected. Phosphorylation of PGC-1α or PGC-1α target molecules can also be measured, for example, by immunoblotting.

Alternatively, modulators of PGC-1α nucleic acid expression (i.e., compounds which can be used to treat a disorder characterized by aberrant or abnormal PGC-1α nucleic acid expression or PGC-1α protein activity) can be identified in a method wherein a cell is contacted with a candidate compound and the expression of PGC-1α mRNA or protein in the cell is determined. The level of expression of PGC-1α mRNA or protein in the presence of the candidate compound is compared to the level of expression of PGC-1α mRNA or protein in the absence of the candidate compound. The candidate compound can then be identified as a modulator of PGC-1α nucleic acid expression based on this comparison and be used to treat a disorder characterized by aberrant PGC-1α nucleic acid expression. For example, when expression of PGC-1α mRNA or polypeptide is greater (statistically significantly greater) in the presence of the candidate compound than in its absence, the candidate compound is identified as a stimulator of PGC-1α nucleic acid expression. Alternatively, when PGC-1α nucleic acid expression is less (statistically significantly less) in the presence of the candidate compound than in its absence, the candidate compound is identified as an inhibitor of PGC-1α nucleic acid expression. The level of PGC-1α nucleic acid expression in the cells can be determined by methods described herein for detecting PGC-1α mRNA or protein.

Modulators of PGC-1α protein activity and/or PGC-1α nucleic acid expression identified according to these drug screening assays can be used to treat, for example, type I muscle associated disorders such as heart failure, disuse atrophy, mitochondrial myopathies, and/or systemic metabolic diseases. Modulators of PGC-1α protein activity and/or PGC-1α nucleic acid expression may also be used to treat disorders related to other functions of PGC-1α unrelated to type I muscle formation. These methods of treatment include the steps of administering the modulators of PGC-1α protein activity and/or nucleic acid expression, i.e., in a pharmaceutical composition as described in subsection IV above, to a subject in need of such treatment, i.e., a subject with a disorder described herein.

This invention is further illustrated by the following examples which should not be construed as limiting. The contents of all references, patent applications, patents, and published patent applications, as well as the Figures and the Sequence Listing cited throughout this application are hereby incorporated by reference.

### EXAMPLES

### Example 1

### PGC-1α is Preferentially Expressed in Slow Twitch Muscle Fibers

This example describes the investigation of PGC-1α expression levels in muscle. RNA was extracted from various types of mouse muscle using standard methods and subjected to a standard Northern blotting protocol using a PGC-1α probe. High levels of PGC-1α mRNA expression were seen in soleus (slow-twitch muscles). Extensor digitorum longus (EDL), quadriceps, gastrocnemius, and tibialis anterior (TA) muscles (all fast-twitch muscles) all showed low-level expression. PGC-1 (expression was also examined in soleus, EDL, quadriceps, and TA muscles. Moderate expression was seen in all of these muscle types.

### Example 2

### Induction of Slow-Twitch Muscle Fiber Differentiation by Transgenic Overexpression of PGC-1α

This example describes the results of overexpression of PGC-1α in transgenic mice. The PGC-1α cDNA sequence was placed under the control of a muscle-specific promoter (the muscle creatine kinase (MCK) promoter). The muscle creatine kinase promoter is expressed in both type I and type II muscle fibers, but is enriched in type II muscle. Transgenic mice were generated using DNA microinjection and screened by PCR. Four independent founder lines were obtained (#23, #26, #29, #31) and mated with wild type mice to obtain progeny for use in experiments. Transgenic lines #23 and #31 showed strong PGC-1α mRNA expression. Line #26 showed low-level PGC-1α mRNA expression. Line #29 showed little PGC-1α mRNA expression. Western blotting showed that PGC-1α protein expression levels were not increased in the soleus (type I) muscles of the high expressing transgenic mice. While no expression of PGC-1α protein is normally seen in plantaris muscles in non-transgenic mice, PGC-1α protein is expressed in the plantaris muscles of the high-expressing transgenic mice at the same level as in soleus muscle.

mRNA was extracted from the muscle fibers of the transgenic lines and subjected to Northern blotting. Transgenic PGC-1α expression resulted in enhanced expression of markers indicative of mitochondrial biogenesis. These markers include medium chain Acyl CoA dehydrogenase (MCAD), cytochrome c oxidase II (COX II), cytochrome c oxidase IV (COX IV), and cytochrome c. Transgenic PGC-1α expression also resulted in a dose-dependant decrease in the expression of a type II (fast-twitch) fiber specific marker (troponin I fast) and a dose-dependant increase in the expression of type I (slow-twitch) fiber specific markers (myoglobin, troponin I slow) in otherwise fast-twitch (type II) fibers, indicating a switch of differentiation program toward type I fibers in the presence of PGC-1α. Western blotting also indicated a switch in the differentiation program toward type I fibers in the presence of PGC-1α. The vastus muscle isolated from the transgenic mice showed strong increases in the levels of myosin, troponin, and cytochrome c (a mitochondrial marker) protein.

Macroscopic examination of the skeletal muscles of the transgenic mice indicated a greatly increased amount of dark-colored (type I) muscle throughout the entire bodies of the mice, as compared to wild type. Specific examination of the hind-limb muscles further showed that the muscles were much darker than the same muscles in the wild type mice. Specific side-by-side examination of the soleus and gastrocnemius muscles showed that the gastrocnemius muscle (normally a type II muscle) was the same dark color in the transgenic mice as the soleus muscle. Metachromatic and anti-myosin histological analysis of plantaris muscle confirmed that the number of type I fibers is significantly increased in the transgenic mice, as compared to their wild type littermate controls.

The muscles from the transgenic mice were also tested for type I specific functional properties. EDL muscles were isolated and subjected to eletrostimulation, and the force generated by the muscles was measured. Fatigue was defined as the time point when the force generated dropped to 30% of the initial force generated. This assay mimics the effects of exercise on the muscles. Using this assay, the EDL muscles isolated from the transgenic mice are significantly more resistant to exercise-induced fatigue (P<0.05), a hallmark for slow-twitch muscle fibers and muscles following endurance training.

### Example 3

### Autoregulatory Loop Controls PGC-1α Expression in Skeletal Muscle

Skeletal muscle contains muscle fibers that differ greatly in their oxidative capacity. Prolonged electrical stimulation or exercise training can lead to a muscle fiber type conversion of type II (fast-twitch) to type I (slow-twitch) fibers (Booth, F. W., and Thomason, D. B. (1991) Physiol Rev 71, 541-585). Conversely, physical inactivity or denervation can cause a switch to type II fibers (Booth, F. W., and Thomason, D. B. (1991) Physiol Rev 71, 541-585). The conversion to type I fibers is characterized by a dramatic change in expression of a large number of genes that increase the oxidative capacity and number of mitochondria, as well as synthesis of distinct contractile proteins characteristic of this muscle fiber type (Berchtold, M. W., et al. (2000) Physiol Rev 80, 1215-1265). Exercise training is accompanied by an increase in motor nerve activity that subsequently elevates intracellular calcium levels in the muscle (Olson, E. N., and Williams, R. S. (2000) Bioassays 22, 510-519; Hood, D. A. (2001) J Appl Physiol 90, 1137-1157). Calcium and the calcium-binding protein calmodulin activate both the calcium/calmodulin-dependent protein kinase IV (CaMKIV) and the protein phosphatase calcineurin A (CnA) as well as many other factors (Hood, D. A. (2001) J Appl Physiol 90, 1137-1157). Activated CaMKIV catalyzes protein phosphorylation events that result in release of the myocyte enhancer factor 2 (MEF2) transcription factors from a complex including the histone deacetylases HDAC1/2 and HDAC4/5, the repressor Cabin-1 and the adaptor mSin3 (Corcoran, E. E., and Means, A. R. (2001) J Biol Chem 276, 2975-2978). Upon phosphorylation by CaMKIV, these factors are bound to 14-3-3 proteins and exported from the nucleus; as a consequence, the MEF2s are now transcriptionally active and can bind co-activator proteins including CBP/p300 or PGC-1α (McKinsey, T. A., et al. (2001) Curr Opin Genet Dev 11, 497-504; McKinsey, T. A., et al. (2002) Trends Biochem Sci 27, 4047; Michael, L. F., et al. (2001) Proc Natl Acad Sci USA 98, 3820-3825).

In another arm of the calcium signaling pathway, activated CnA dephosphorylates members of the nuclear factor of activated T-cells (NFAT) family, thereby stimulating a cytoplasmic-nuclear translocation of these proteins (Olson, E. N., and Williams, R. S. (2000) Cell 101, 689-692). The combined action of MEF2s and NFATs in the nucleus increases the transcription of prototypical muscle fiber type I genes, and thus promotes muscle fiber type switching from type II to type I (Chin, E. R., et al. (1998) Genes Dev 12, 2499-2509). Activated CnA provides a further boost to this process by dephosphorylating MEF and enhancing its transcriptional activity (Wu, H., et al. (2001) EMBO J 20, 6414-6423).

Proof of concept of this general model came from transgenic mice that express either constitutively active CnA or CaMKIV, respectively (Naya, F. J., et al. (2000) J Biol Chem 275, 4545-4548; Wu, H., et al. (2002) Science 296, 349-352). In these mice, the relative amount of type I muscle fibers is greatly increased in comparison to wildtype animals, supporting a crucial role for CnA and CaMKIV in muscle fiber type determination. They are furthermore characterized by enhanced mitochondrial biogenesis, upregulation of enzymes involved in oxidative metabolism and greater resistance to fatigue (Wu, H. et al. (2002) Science 296, 349-352). Interestingly, the main effect of CaMKIV and CnA was observed in an increase of type I muscle fiber number, but not skeletal muscle hypertrophy. Although CnA has been implicated in the molecular mechanism that stimulates hypertrophy, these animal models demonstrate that slow fiber type determination and muscular hypertrophy can be separated and depend on the cellular stimuli and context (Naya, F. J., et al. (2000) J Biol Chem 275, 4545-4548; Musaro, A., et al. (1999) Nature 400, 581-585).

PGC-1α was originally cloned from brown adipose tissue and has been shown to coactivate a variety of nuclear receptors and other transcription factors (described in U.S. Pat. No. 6,166,192, incorporated herein in its entirety by reference). Moreover, PGC-1α is a potent stimulator of mitochondrial biogenesis and oxidative metabolism in several tissues including skeletal muscle (Michael, L. F., et al. (2001) Proc Natl Acad Sci USA 98, 3820-3825; Wu, Z., et al. (1999) Cell 98, 115-124). These aspects of energy metabolism are crucial in muscle fiber type differentiation, and thus, as set forth herein, transgenic expression of PGC-1α driven by a muscle-specific promoter results in a dramatic increase of type I muscle fibers. Increased expression of fiber type I proteins, higher oxidative capacity and greater resistance to fatigue can be observed in the mice that ectopically express PGC-1α. It has also been found that PGC-1α may regulate its own transcription and with this autoregulatory loop helps to maintain expression of fiber type I-specific genes.

### Methods and Materials

Plasmids and Reagents.

The 5′-flanking sequence of mouse PGC-1α was obtained from the CELERA™ Mouse Genome database. Various fragments of this promoter were subsequently amplified by PCR and subcloned into the pGL3basic reporter gene vector (PROMEGA™). Thus, the constructs containing the regions between +78 and −2533 or −6483 in respect to the transcriptional start site were denominated 2 kb and 6 kb, respectively. All constructs were verified by sequencing. Expression plasmids for MEF2C, MEF2D, NFATc3, CaMKIV and constitutively active CnA were gifts from Dr. Eric N. Olson, University of Texas Southwestern Medical Center, Dallas, Tex. The dominant negative cyclic AMP response element binding protein (CREB) called ACREB was provided by Dr. Charles Vinson, National Cancer Institute, National Institutes of Health, Bethesda, Md. All reagents were obtained from SIGMA™.

Site-Directed Mutagenesis.

Site-directed mutagenesis was performed as described previously (Handschin, C., and Meyer, U. A. (2000) J Biol Chem 275, 13362-13369). Briefly, PCR amplifications were performed by using overlapping primers at the target sites, the resulting PCR product was digested with DpnI to remove residual template and subsequently transformed into bacteria. Clones containing the mutation were digested with KpnI and BglII and the insert subcloned into a new reporter gene vector. The cAMP-responsive element (CRE) and the MEF-binding site were mutated into a BglII and a SacII site and termed ΔCRE and ΔMEF2, respectively. Constructs were verified by both restriction digestion and sequencing.

Cell Culture, Transfection and Reporter Gene Assays.

C2C12 cells were maintained in DMEM supplemented with 10% fetal calf serum and 1 μM Na-pyruvate in subconfluent cultures. Cells were subsequently transfected using Lipofectamine transfection reagent (INVITROGEN™) and reporter gene levels were determined 48 hours after transfection. Cells were lysed and analyzed for luciferase expression using the Enhanced Luciferase Assay Kit (BD PHARMINGEN™) according to the supplier's manual. Reporter gene expressions were subsequently normalized against β-galactosidase levels driven by the cotransfected pSV-β-galactosidase expression vector (PROMEGA™). Finally, these relative expression were normalized against empty reporter gene vector expression.

Analysis of PGC-1α Gene Expression in Wildtype and Transgenic PGC-1α Mice.

Wildtype and transgenic mice from strain #31 (described above in Example 2) that highly express PGC-1α in muscle were sacrificed, skeletal muscle was collected, total RNA isolated using the Trizol reagent following the manufacturer's instructions and subsequently reverse transcribed. Primers for the ABI Prism 7700 sequence detector (APPLIED BIOSYSTEMS™) were designed with the Primer Express™ software targeting either PGC-1α exon 2, PGC-1α 3′ untranslated region, mouse cytochrome c, uncoupling protein 3, myoglobin, glyceraldehyde 3-phosphate dehydrogenase and 18S rRNA. Using the SYBR green PCR master mix, expression levels of total PGC-1α (primers for exon 2) and endogenous PGC-1α (primers for the 3′ untranslated region) as well as of the other genes were determined from at least three wildtype and transgenic mice and subsequently normalized against 18S rRNA levels.

### Results

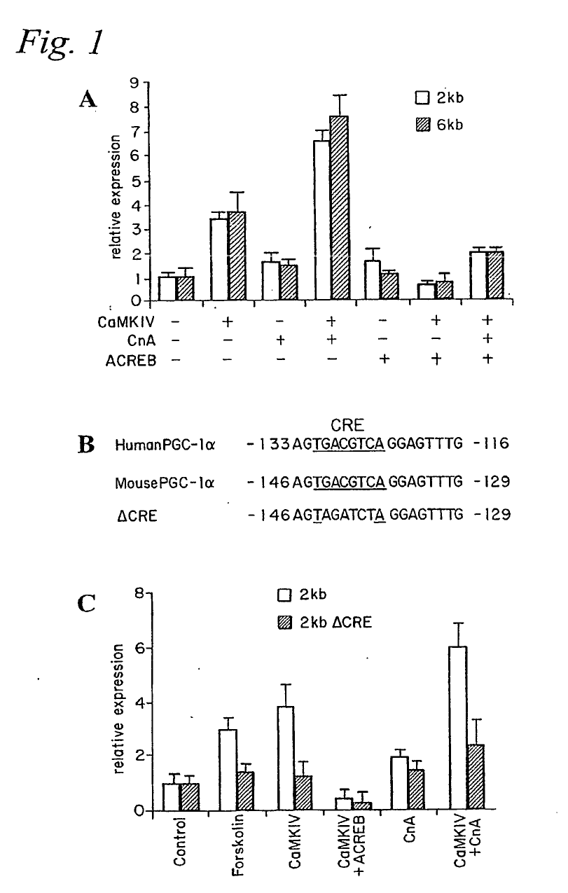

PGC-1α has been shown to be elevated in the skeletal muscles of mice that contain CaMKIV expressed transgenically in this tissue (Wu, H., et al. (2002) Science 296,349-352). Furthermore, CaMKIV was found to activate the human PGC-1α promoter but the mechanistic basis to this has not been investigated (Wu, H., et al. (2002) Science 296, 349-352). As depicted in FIG. 1A, proximal promoter fragments that are 2 kb or 6 kb in size are both activated when co-transfected with a vector expressing a constitutively active CaMKIV. Coexpression of a constitutively active CnA has only a minimal effect on reporter gene levels corroborating the results obtained in the transgenic CaMKIV and CnA models, respectively (Wu, H., et al. (2002) Science 296, 349-352). The combination of CaMKIV and CnA has at least an additive effect in increasing transcription controlled by the PGC-1α promoter. In this experiment, C2C12 cells were cotransfected with expression plasmids for CnA, CaMKIV and ACREB together with reporter gene plasmids containing different fragments of the mouse PGC-1α promoter. After 48 hours, cells were harvested and reporter gene levels determined.

CaMKIV has been shown to phosphorylate and activate many proteins including CREB. Since CREB has been shown to be an important component of PGC-1α expression in the fasted liver (Herzig, S., et al. (2001) Nature 413, 179-183), dominant negative ACREB protein was utilized to examine a potential role for CREB in the CaMKIV-mediated control of the PGC-1α promoter. While ACREB had no effect on the PGC-1α promoter when expressed alone, this protein is able to severely reduce the activation of the PGC-1α promoter by CaMKIV alone or CaMKIV in combination with CnA (FIG. 1A).

The human PGC-1α promoter contains a CRE at −133/−116 that is crucial for PGC-1α induction by cAMP in the liver (Herzig, S., et al. (2001) Nature 413, 179-183). Similarly, a very conserved putative CRE can be identified in the mouse PGC-1α promoter at approximately the same distance from the transcriptional start site (FIG. 1B). The functional role of this mouse CRE was, tested by site-directed mutagenesis followed by stimulation of the mutated promoter with 100 μM forskolin, a reagent which stimulates formation of cAMP, for 10 hours. As shown in FIG. 1C, mutagenesis of the CRE site abolished induction of 2 kb of the mouse PGC-1α promoter by forskolin. The same results were obtained when treating the cells with 8-bromo-cAMP whereas the inactive analog 1,9-dideoxyforskolin had no effect on the PGC-1α promoter (data not shown). Importantly, the CRE PGC-1α promoter showed dramatically impaired response to CaMKIV alone or the combination of CaMKIV and CnA in these assays, indicating a key role for CREB in the induction of PGC-1α expression by these mediators of calcium signaling (FIG. 1C). Similar observations were made when using larger fragments of the mouse PGC-1α promoter. In this experiment, C2C12 cells were cotransfected with expression plasmids for CnA, CaMKIV and ACREB together with reporter gene plasmids containing 2 kb of wildtype or PGC-1α promoter with a mutation in the CRE site (ACRE), respectively. Cells were subsequently treated with either vehicle (0.1% DMSO) or 100 μM forskolin for 10 hours and harvested 48 hours after transfection before reporter gene levels were determined.

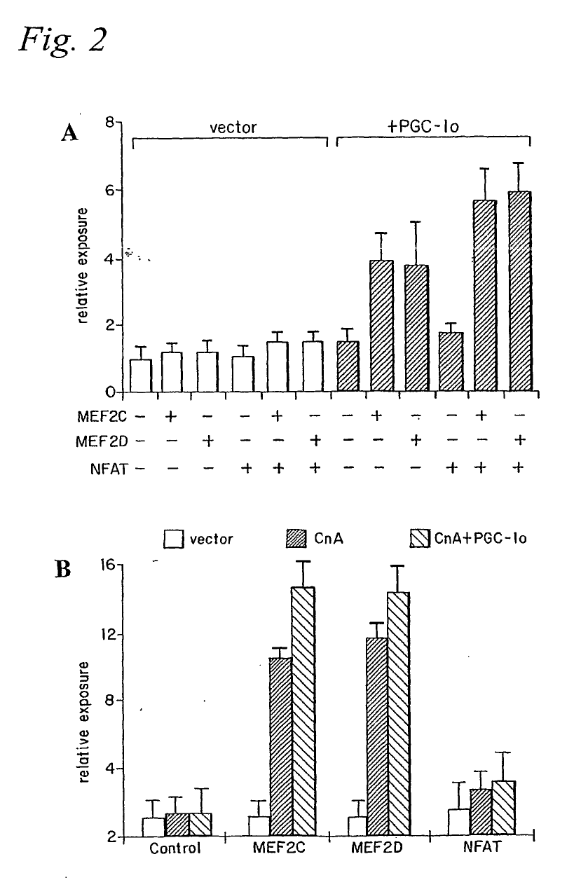

While CREB appears to be an important factor in the induction of PGC-1α, the increased effect of CaMKIV in combination with CnA indicates that factors in addition to CREB are likely to be involved in the transcription of the PGC-1α gene in muscle. Since MEF2 and NFAT transcription factors are known targets of CaMKIV and CnA in muscle fiber type determination, the role of these factors in control of the PGC-1α promoter was tested. As depicted in FIG. 2A, MEF2C, MEF2D or NFATc3 alone did not have a significant effect on the 6 kb PGC-1α promoter construct. However, since MEF2 proteins are known to be coactivated by PGC-1α (Michael, L. F., et al. (2001) Proc Natl Acad Sci USA 98, 3820-3825), these factors were cotransfected and the experiments revealed coactivation of both MEF2C and MEF2D but not NFAT by PGC-1α (FIG. 2A). These data indicate that PGC-1α participates in the activation of its own promoter, and the MEF2 proteins may be upstream as well as downstream of PGC-1α expression. In this experiment, C2C12 cells were cotransfected with expression plasmids for MEF2C, MEF2D, NFAT and PGC-1α together with reporter gene plasmids containing 6 kb of the mouse PGC-1α promoter. After 48 hours, cells were harvested and reporter gene levels determined.

The transcriptional capacities of both MEF2 and NFAT are known to be increased by CaMKIV- and CnA-mediated changes in phosphorylation status. CnA is able to substantially increase the activity of MEF2C and MEF2D (FIG. 2B). The strongest effect on the PGC-1α promoter was observed when cotransfecting MEF2C or MEF2D together with CnA and PGC-1α (FIG. 2B). No effect was found by the coexpression of any of these proteins with NFAT. In contrast to the effects of CnA, the effect of CaMKIV on this reporter gene construct was neither changed by addition of MEF2s nor PGC-1α. This indicates that a major effect of CaMKIV may be in activating PGC-1α expression via CREB independent on PGC-1α coactivation whereas CnA apparently is able to further increase the potency of MEF2s to stimulate transcription of PGC-1α. Similarly, in this experiment, C2C12 cells were cotransfected with expression plasmids for MEF2C, MEF2D, NFAT, CnA and PGC-1α together with reporter gene plasmids containing 6 kb of the mouse PGC-1α promoter. After 48 hours, cells were harvested and reporter gene levels determined.

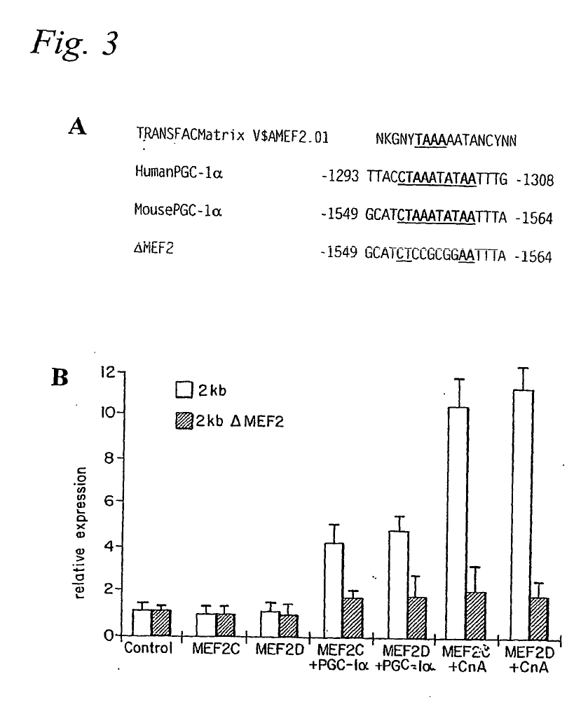

As depicted in FIG. 3A, computer-aided sequence analysis of the mouse PGC-1α 5′-flanking region revealed a high-scoring MEF2 binding site at −1464/−1447 (TRANSFAC matrix V$AMEF2.01) and a NFAT binding site at −1547/−1536 (TRANSFAC matrix V$NFAT.01) (Quandt, K., et al. (1995) Nucleic Acids Res 23, 4878-4884). Similar configurations of adjacent MEF2 and NFAT binding sites have previously been described in several muscle fiber type I specific promoters (Chin, E. R., et al. (1998) Genes Dev 12, 2499-2509). Thus, whether site-directed mutagenesis of this site affects MEF2 activity on the reporter gene construct was tested. The mutated 2 kb fragment (referred to as ΔMEF2) is no longer able to mediate MEF2C or MEF2D induction either when activated by CnA or when coactivated with PGC-1α, indicating that this site is responsible for the MEF2 action (FIG. 3B). In this experiment, C2C12 cells were cotransfected with expression plasmids for MEF2C, MEF2D, CnA and PGC-1α together with reporter gene plasmids containing 2 kb of wildtype or 2 kb of mouse PGC-1α promoter with a mutation in the MEF2-binding site (ΔMEF2). After 48 hours, cells were harvested and reporter gene levels determined.

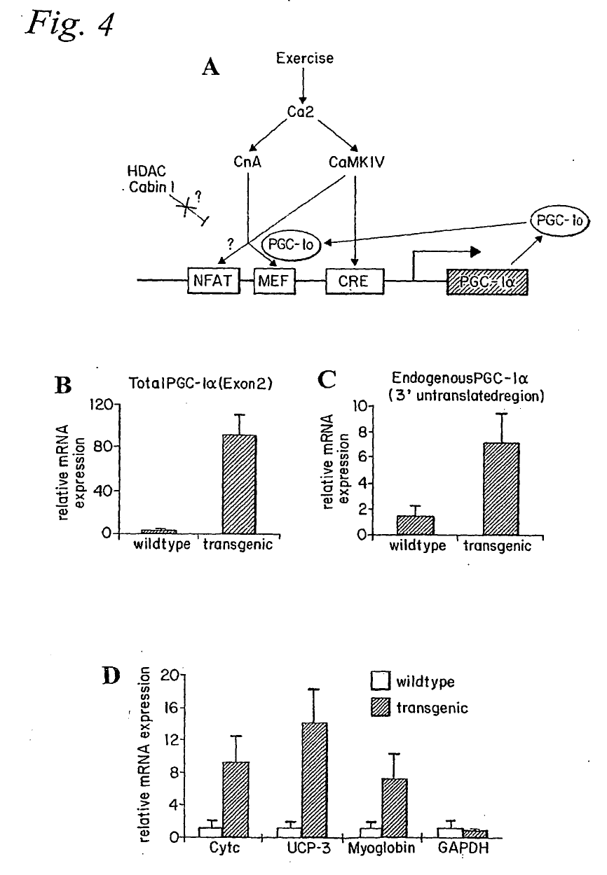

The ability of PGC-1α to stimulate the PGC-1α promoter via coactivation of the MEF2 proteins indicates a potential autoregulatory loop (FIG. 4A). Exercise and subsequently elevated intracellular calcium levels result in an activation of both CaMKIV and CnA in skeletal muscle. Activated CaMKIV can phosphorylate CREB which then increases transcription of PGC-1α via a conserved CREB-binding site in the proximal promoter. Moreover, CaMKIV and CnA activate the transcriptional activity of MEF2s in part by promoting the dissociation of inhibitory HDACs and Cabin1. MEF2s, potentially in combination with NFAT, bind to at least one MEF2-binding site in the PGC-1α flanking region and increase transcriptional activity. Newly synthesized PGC-1α protein can coactivate MEF2s and thus positively regulate its own transcription. PGC-1α may also compete with the inhibitory HDACs and Cabin 1 for binding to MEF2s and thus ensure a stable transcription leading to muscle fiber type I determination.

Thus, increased levels of PGC-1α protein should lead to a stable expression of PGC-1α by coactivation of MEF2s on its own promoter. In order to critically test this hypothesis, real-time PCR primers for the PGC-1α 3′ untranslated region were designed that should allow distinct determination of the levels of ectopically expressed and endogenous PGC-1α. Total RNA from wildtype and transgenic skeletal muscle were analyzed for the expression levels of total and endogenous PGC-1α mRNA using real-time PCR primers targeted for exon 2 (FIG. 4B) and the 3′ untranslated region (FIG. 4C), respectively. The same RNA was analyzed for the expression of cytochrome c (Cyt c), uncoupling protein-3 (UCP-3), myoglobin and glyceraldehyde 3-phosphate dehydrogenase (GAPDH) (FIG. 4D). Relative mRNA expression levels were normalized against 18S rRNA levels.

As shown in FIG. 4B, primers designed to target PGC-1α exon 2 reveal a more than 80-fold increase in total PGC-1α levels in the muscle of transgenic mice in comparison to wildtype animals. These findings are similar to the results observed in Northern blots for the high-expressing transgenic line #31 (Lin, J., et al. (2002) Nature 418, 797-801). When exclusively measuring endogenous PGC-1α with primers designed for the 3′ untranslated region that is missing in the transgenic constructs, an approximately 7-fold elevation of endogenous PGC-1α was observed in the transgenic animals as compared to wildtype mice (FIG. 4C). A robust increase in the transcript levels of cytochrome c (Cyt c), uncoupling protein-3 (UCP-3) and myoglobin in the RNA isolated from skeletal muscle of the transgenic mice was also shown whereas glyceraldehyde 3-phosphate dehydrogenase (GAPDH) levels remained unchanged (FIG. 4D).

These results strongly indicate a regulation of PGC-1α transcription by PGC-1α protein in an autoregulatory loop. Moreover, PGC-1α gene expression in muscle is reminiscent of other prototypical fiber type I genes such as myoglobin. Thus, gene expression analysis of transgenic PGC-1α animals further underscores the importance of PGC-1α in its own regulation.

Accordingly, based on these results, it appears that CaMKIV stimulates PGC-1α expression, namely by phosphorylating and thus activating CREB, a transcription factor implicated in PGC-1α transcription in many different tissues (Puigserver, P. et al. (1998) Cell 92, 829-839; Herzig, S., et al. (2001) Nature 413, 179-183).

These data indicate an initial activation of PGC-1α transcription by CaMKIV via CREB (FIG. 4A). As soon as PGC-1α is expressed, it can act as cofactor for de-repressed MEF2 on fiber type I target genes as well as its own promoter, thus ensuring stable, high expression levels. Moreover, PGC-1α binding to MEF2 may prevent binding of the MEF2-repressing HDACs and Cabin1 proteins. Although CnA and NFAT did not affect the PGC-1α promoter on their own, an increase in CaMKIV- and MEF2-mediated induction was observed when CnA and NFAT were cotransfected. This may be explained by CnA-triggered activation of MEFs and other factors or by stabilization of MEF2-binding to the promoter due to NFAT. Recent reports using in vivo models support the methods described herein, such as the data showing rapid increase of PGC-1α mRNA and protein levels after exercise in rats and man (Goto, M., et al. (2000) Biochem Biophys Res Commun 274, 350-354; Terada, S., et al. (2002) Biochem Biophys Res Commun 296, 350-354; Baar, K., et al. (2002) FASEB J 16, 1879-1886).

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for modulating type I muscle formation comprising contacting a cell with an agent that modulates PGC-1α expression or activity, such that type I muscle formation is modulated.

2. The method of claim 1, wherein PGC-1α expression or activity is increased.

3. The method of claim 1, wherein PGC-1α expression or activity is decreased.

4. The method of claim 1, wherein type I muscle formation is increased.

5. The method of claim 1, wherein the agent is a PGC-1α nucleic acid molecule.

6. The method of claim 5, wherein the PGC-1α nucleic acid molecule is derived from a human.

7. The method of claim 6, wherein the PGC-1α nucleic acid molecule comprises the nucleic acid sequence of SEQ ID NO:1.

8. The method of claim 5, wherein the PGC-1α nucleic acid molecule is contained within a vector.

9. The method of claim 8, wherein the vector is an adenoviral or an adeno-associated vector.

10. The method of claim 1, wherein the agent is a PGC-1α polypeptide.

11. The method of claim 10, wherein the PGC-1α polypeptide is derived from a human.

12. The method of claim 11, wherein the PGC-1α polypeptide comprises the amino acid sequence of SEQ ID NO:2.

13. The method of claim 1, wherein the agent is a small molecule.

14. The method of claim 1, wherein the cell is a muscle cell.

15. The method of claim 14, wherein the muscle cell is a skeletal muscle cell.

16. The method of claim 15, wherein the skeletal muscle cell is selected from the group consisting of a type I muscle cell and a type II muscle cell.

17. The method of claim 1, wherein the method is performed in vitro.

18. The method of claim 1, wherein the method is performed in vivo.

19. The method of claim 18, wherein the method is performed in a mouse.

20. The method of claim 18, wherein the method is performed in a human.

21. A method for identifying a compound capable of modulating type I muscle formation comprising:
a) contacting a cell with a compound; and
b) determining whether PGC-1α expression or activity is modulated.

22. The method of claim 21, wherein PGC-1α expression or activity is increased.

23. The method of claim 21, wherein PGC-1α expression is measured by Northern blotting.

24. The method of claim 21, wherein determining whether PGC-1α expression or activity is modulated comprises determining whether expression of at least one of myoglobin, troponin I slow, troponin I fast, MCAD, COX II, COX IV, or cytochrome c is modulated.

25. The method of claim 24, wherein expression is measured by Northern blotting.

26. The method of claim 21, wherein the cell is a muscle cell.

27. The method of claim 21, wherein the muscle cell is a skeletal muscle cell.

28. The method of claim 27, wherein the skeletal muscle cell is selected from the group consisting of: a type I muscle cell and a type II muscle cell.

29. A compound identified by the method of claim 21.

30. A method for identifying a compound capable of treating a disorder characterized by aberrant type I muscle formation comprising assaying the ability of the compound to modulate the expression or activity of PGC-1α to thereby identify a compound capable of treating a disorder characterized by aberrant type I muscle formation.

31. The method of claim 30, wherein PGC-1α expression or activity is increased.

32. The method of claim 30, wherein PGC-1α expression is measured by Northern blotting.

33. The method of claim 30, wherein determining whether PGC-1α expression or activity is modulated comprises determining whether expression of at least one of myoglobin, troponin I slow, troponin I fast, MCAD, COX II, COX IV, or cytochrome c is modulated.

34. The method of claim 33, wherein expression is measured by Northern blotting.

35. The method of claim 30, wherein the cell is a muscle cell.

36. The method of claim 35, wherein the muscle cell is a skeletal muscle cell.

37. The method of claim 36, wherein the skeletal muscle cell is selected from the group consisting of: a type I muscle cell and a type II muscle cell.

38. A compound identified by the method of claim 30.

39. A method for treating a subject having a disorder characterized by aberrant type I muscle formation comprising administering to the subject an agent capable of modulating PGC-1α expression or activity, such that the disorder is treated.

40. The method of claim 39, wherein the disorder is selected from the group consisting of heart failure, disuse atrophy, a mitochondrial myopathy, and a systemic metabolic disorder.

41. The method of claim 39, wherein PGC-1α expression or activity is increased.

42. The method of claim 41, wherein type I muscle formation is increased.

43. The method of claim 39, wherein the agent is a PGC-1α nucleic acid molecule.

44. The method of claim 43, wherein the PGC-1α nucleic acid molecule is derived from a human.

45. The method of claim 44, wherein the PGC-1α nucleic acid molecule comprises the nucleic acid sequence of SEQ ID NO:1.

46. The method of claim 43, wherein the PGC-1α nucleic acid molecule is contained within a vector.

47. The method of claim 46, wherein the vector is an adenoviral or an adeno-associated vector.

48. The method of claim 39, wherein the agent is a small molecule.

49. A method for increasing type I muscle formation in a subject comprising administering to the subject an agent capable of increasing PGC-1α expression or activity, such that type I muscle formation is increased.

50. The method of claim 49, wherein the agent is a PGC-1α nucleic acid molecule.

51. The method of claim 50, wherein the PGC-1α nucleic acid molecule is derived from a human.

52. The method of claim 51, wherein the PGC-1α nucleic acid molecule comprises the nucleic acid sequence of SEQ ID NO:1.

53. The method of claim 50, wherein the PGC-1α nucleic acid molecule is contained within a vector.

54. The method of claim 53, wherein the vector is an adenoviral or an adeno-associated vector.

55. The method of claim 49, wherein the agent is a small molecule.

56. A nonhuman transgenic animal comprising an exogenous PGC-1α nucleic acid molecule, wherein the exogenous PGC-1α nucleic acid molecule is expressed in the skeletal muscle of the animal.

57. The transgenic animal of claim 56, wherein the exogenous PGC-1α nucleic acid molecule is operatively linked to a muscle specific promoter.

58. The transgenic animal of claim 57, wherein the muscle specific promoter is selected from the group consisting of: the muscle creatine kinase promoter, the dystrophin promoter, the myostatin promoter, the GDF-8 promoter, the UCP-3 promoter, the MyoD promoter, the MEF2 the promoter, the myosin heavy chain promoter, the myosin light chain promoter, and a troponin promoter.

59. The transgenic animal of claim 57, wherein the expression of at least one of myoglobin, troponin I slow, MCAD, COX II, COX IV, or cytochrome c is upregulated in the muscle cells of the animal.

60. The transgenic animal of claim 56, wherein the animal is a mouse.
